(19) United States
(12) Patent Application Publication
(10) Pub. No.: US 2023/0109468 A1
(43) Pub. Date: Apr. 6, 2023

(54) Fabrication-Free Microfluidic Device for Scalable, High-Volume Bacterial Electroporation

(71) Applicant: Massachusetts Institute of Technology, Cambridge, MA (US)

(72) Inventors: Po-Hsun Huang, Quincy, MA (US); Cullen Richard Buie, Cambridge, MA (US); Sijie Chen, Cambridge, MA (US)

(21) Appl. No.: 17/935,844

(22) Filed: Sep. 27, 2022

Related U.S. Application Data
(60) Provisional application No. 63/248,696, filed on Sep. 27, 2021.

(51) Int. Cl. C12M 1/42; C12N 13/00; C12N 15/87; C12M 1/00

(52) U.S. Cl. CPC C12M 35/02; C12N 13/00; C12N 15/87; C12M 29/00; C12M 1/42

(57) ABSTRACT

A disposable, fabrication-free, high-volume electroporation device may process cell samples of large volume without compromising transformation efficiency and cell viability, while precipitously reducing the entire processing time and effort. An embodiment includes at least two hollow, tubular conductive elements and an insulating structure, defining a channel, that fluidically couples the at least two conductive elements to define an electroporation flow path in the channel for flow-through electroporation. The high-volume electroporation device can be an alternative to cuvettes for typical volume electroporation, but can also be an indispensable tool to process large volume samples for applications, such as creation of modified sample libraries.

### RELATED APPLICATION

This application claims the benefit of U.S. Provisional Application No. 63/248,696, filed on Sep. 27, 2021, the entire teachings of which are incorporated herein by reference.

### GOVERNMENT SUPPORT

This invention was made with government support under GM135102 awarded by the National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
a) File name: 00502359001_Replacement.xml; created Oct. 18, 2022, 11,243 Bytes in size.

### BACKGROUND

One of the key steps in bacterial genetic engineering is the delivery of DNA into cells, which can be realized by mechanical, chemical, or electrical methods [1-3]. Among these methods, electroporation has been the gold standard because it is not cell-type-specific [2], can deliver molecules of various sizes [4], and can exhibit relatively high efficiency under optimized conditions [2, 5]. For optimal electric field conditions, genetic material enters cells through reversible pores formed in the cell membrane [6, 7]. Electroporation is typically performed using cuvettes, in an operator-dependent manner that is limited to small batches of volume 1 mL or less. Even with high efficiency, creation of a comprehensive mutant library with hundreds of thousands of mutants [8-10] for functional-genomics studies can require electroporation of large volumes (tens of milliliters) of saturated bacterial culture, which corresponds to hundreds of cuvette-based electroporation reactions. Performing serial electroporation with manual pipetting is a labor-intensive, time-consuming, and costly process. Moreover, cuvette-based electroporation suffers from issues such as residual volume and joule heating [11, 12], which affect electroporation efficiency, cell viability, and overall yield.

Performing electroporation in a microfluidic format [11-14] can remove the need for manual pipetting and improve heat dissipation [11, 14], thereby increasing electroporation efficiency and cell viability. However, most microfluidic devices involve complicated fabrication processes using PDMS [15-19] (polydimethylsiloxane, called PDMS or dimethicone, is a polymer widely used for the fabrication and prototyping of microfluidic chips), which is an obstacle to widespread adoption, particularly within the microbiology community that would most benefit.

Microfluidics-based electroporation devices are also typically limited by the sample volume they can handle. These devices are commonly used for mammalian cells [18, 20], with just a few examples of applications to bacteria [19, 21]. Several commercial products [22-26] have demonstrated the potential for scaling up electroporation to throughput of up to ˜100 mL at 8 mL/min [26], but most have been applied only to mammalian cells and still rely on batch-wise operation [22-26]. Moreover, existing commercial systems require sophisticated electroporation chambers that limit the volume that they can process. Thus, the capabilities of these systems for large-volume bacterial electroporation are yet unproved.

### SUMMARY

An ideal genetic transformation system would allow for a wide range of sample volumes to accommodate different applications, especially involving the creation of mutant libraries given the low electroporation efficiency of many understudied yet health-relevant bacterial species [10, 27, 28]. A scalable, high-volume electroporation device should be easily assembled by a microbiologist without sophisticated fabrication, compatible with commercially available and common laboratory equipment, and able to process relevant sample volumes in minutes to minimize biological variability. To this end, disclosed herein is a simple yet powerful Microfluidic Tubing-based Bacterial Electroporation device (M-TUBE) that enables flexible electroporation of large-volume bacterial samples. M-TUBE facilitates scalable, continuous flow, large-volume bacterial electroporation without the need for micro/nanofabrication, PDMS casting, or 3D printing of microfluidic channels and electrodes.

Example embodiments of an electroporation device are described herein that can provide for scalable, high-throughput, flow-through electroporation.

An example embodiment of the electroporation device includes at least two conductive elements, each of the at least two conductive elements being of a hollow, tubular structure. The device further includes an insulating structure defining a channel. The insulating structure is configured to couple the at least two conductive elements fluidically such that, in coupled arrangement, the at least two conductive elements and the insulating structure define an electroporation flow path in the channel for flow-through electroporation.

A method of fabricating an electroporation device according to an embodiment of the electroporation device includes inserting a conductive element at each opposing end of an insulating structure defining a channel. Each conductive element is of a hollow, tubular structure. The conductive elements and the insulating structure, in coupled arrangement, define an electroporation flow path in the channel for flow-through electroporation.

A kit includes a plurality of conductive elements and a plurality of insulating structures.

The channel defined by the insulating structure can be of a constant diameter. For example, the channel can be a constant diameter channel extending between the at least two conductive elements. The channel can be configured to enable fluid to travel through the electroporation flow path at a velocity of about 0.1 m/s to about 5 m/s, to travel through the electroporation flow path at a constant velocity, or a combination thereof.

Each of the conductive elements can be a cannula comprising a conductive material, for example a syringe needle or portion thereof. The insulating structure can be, for example, a polymer tube. The insulating structure can be configured to receive the conductive elements as inserts at opposing ends of the channel. To provide for ease of assembly, the insulating structure can include markings indicating an insertion distance of each the at least two conductive elements, stops defining an insertion distance of each the at least two conductive elements, or a combination thereof. In an alternative embodiment, the conductive elements can include markings indicating an insertion distance of each the at least two conductive elements, stops defining an insertion distance of each the at least two conductive elements, or a combination thereof.

The at least two conductive elements, insulating structure, or combinations thereof may be interchangeable structures with fixed geometries that may be designed to provide for different fixed separation distances or adjustable distances. The insulating structure may be formed of a polymer or other insulating material tubing or substituted with other types of insulating spacers, including those with a tubular channel drilled therethrough.

In some embodiments, the conductive elements can be inserted into the channel of the insulating structure with a gap therebetween of about 1 mm to about 50 mm, or of about 1 mm to about 10 mm. The channel defined by the insulating structure can have a diameter of about 0.1 mm to about 5 mm.

The electroporation device can further include a fluid pump coupled to an upstream one of the at least two conductive elements. The fluid pump can be configured to supply a cell media to the channel at a flow rate of about 1 mL/min to about 1500 mL/min, or of about 1 mL/min to about 100 mL/min. A controller in operative arrangement with the fluid pump can be configured to control the flow rate based upon a selected residence time of cells exposed to an electric field in the channel.

The electroporation device can further include a power supply in operative arrangement with the at least two conductive elements. A voltage supplied by the power supply can be configured to generate an electric field within the channel of about 0.1 kV/cm to about 100 kV/cm. A controller in operative arrangement with the power supply can be configured to control an applied voltage based upon a selected electric field strength. The fluid pump controller and the power supply controller may be a same controller. An applied voltage can be further based on at least one of a channel diameter and a channel distance. The device can further include an indicator configured to indicate an applied current in the flow path to provide for user feedback that conditions were adequate for electroporation to have successfully occurred.

The insulating structure and the conductive elements can each be disposable or reusable.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic diagram of an assembled electroporation device, also referred to herein as an M-TUBE device.

FIG. 2 is a schematic diagram of an electroporation device with markings.

FIG. 3 is a schematic diagram of an electroporation device with stops.

FIG. 4 is a graph of transformation efficiency (TE) of three tested prototype devices.

FIG. 5A is a schematic diagram of an embodiment of a fabrication-free, microfluidic electroporation device.

FIG. 5B is a photograph of components of the embodiment of the electroporation device of FIG. 5A and a conventional cuvette.

FIG. 5C is a photograph of the embodiment of the electroporation device of FIG. 5A shown in an operative arrangement with a syringe pump for sample delivery.

FIG. 5D is a diagram that depicts a detailed procedure to assemble the embodiment of the electroporation device of FIG. 5A.

FIG. 5E is a diagram of simulation results of electric fields established in the embodiment of the device of FIG. 5A with different inner diameters.

FIG. 5F is a diagram of colony forming units (CFUs) that resulted from flowing cells in the presence and absence of an electric field through the embodiment of the device of FIG. 5A at fluid velocities.

FIG. 5G is a plot of data of transformation efficiencies obtained across the range of flow rates tested in the process of FIG. 5F.

FIGS. 6A-1 through 6D are diagrams of an embodiment of the M-TUBE device and data plots that illustrate that the device exhibits higher efficiency than cuvettes across E. coli strains, is reproducible, and maintains high efficiency across tubing sizes. FIG. 6A-1 is a plot of transformation efficiencies obtained with E. coli strain NEB10β. FIG. 6A-2 is a plot of transformation efficiencies obtained with E. coli strain MG1655. FIG. 6A-3 is a plot of transformation efficiencies obtained with E. coli strain Nissle 1917. FIG. 6B is a diagram illustrating testing methodology. FIG. 6C is a plot of transformation efficiencies obtained demonstrating reproducibility. FIG. 6D is a plot of transformation efficiencies obtained demonstrating scalability.

FIGS. 7A-7C are diagrams that illustrate that M-TUBE efficiently transforms anaerobic bacteria and enables transposon insertion mutagenesis. FIG. 7A is a plot of relative transformation efficiency versus electric field strength of example embodiments of M-TUBE devices as compared with cuvettes. FIG. 7B is a plot of transformation efficiency for M-TUBE as compared with a cuvette. FIG. 7C is a Tri-seq map of transposon insertions resulting from electroporation using M-TUBE.

FIG. 8 is a diagram that illustrates a comparison of transformation efficiency and cell viability obtained using M-TUBE devices and conventional cuvettes.

FIG. 9 is a diagram that illustrates dependence of M-TUBE's electroporation efficiency on the frequency of AC fields applied.

FIG. 10 is a diagram that illustrates a comparison of M-TUBE device performance when the device is supplied with alternating current (AC) fields and direct current (DC) fields.

FIG. 11 is a diagram that illustrates that transformation efficiency is maintained across M-TUBE devices with different diameters.

FIG. 12A and FIG. 12B are diagrams that illustrate a simulated temperature distribution in the microchannel of M-TUBE when cell samples are flowed through the microchannel at a fluid velocity of 148 mm/s (FIG. 12A) and 592 mm/s (FIG. 12B).

FIG. 13A and FIG. 13B are diagrams that illustrate a simulated temperature distribution in the microchannel of M-TUBE when a voltage of 2.25 kV (or 7.50 kV/cm) (FIG. 13A) and 2.00 kV (or 6.67 kV/cm) (FIG. 13B) is applied.

FIG. 14A and FIG. 14B are diagrams that illustrate a simulated temperature distribution in the microchannel of M-TUBE when cell samples with an initial temperature of 20° C. (FIG. 14A) and 4° C. (FIG. 15C) are flowed through the channel.

FIG. 15 is an image of the high-voltage power supply system that may be employed by an embodiment of M-TUBE.

FIG. 16 is a schematic diagram of an example arrangement of electroporation conditions tested in a 96-well deep-well plate.

FIG. 17 is a photograph of M-TUBE set up in an anaerobic chamber.

FIG. 18 is a diagram of a workflow employing a commercial liquid-handling robot for automated liquid transfer and serial dilution.

### DETAILED DESCRIPTION

A description of example embodiments follows.

Conventional cuvette-based and microfluidics-based electroporation approaches for bacterial gene delivery have distinct advantages, but they are typically limited to relatively small sample volumes, reducing their utility for applications requiring high throughput such as the generation of mutant libraries. Disclosed herein are example embodiments of a scalable, large-scale bacterial gene delivery approach enabled by a disposable, user-friendly microfluidic electroporation device requiring minimal device fabrication and straightforward operation. As shown and described, the proposed device can outperform conventional cuvettes in a range of situations, including across Escherichia coli strains with a range of electroporation efficiencies, and we use its large-volume bacterial electroporation capability to generate a library of transposon mutants in the anaerobic gut commensal Bifidobacterium longum. Example embodiments of the disposable, user-friendly microfluidic electroporation device are described below.

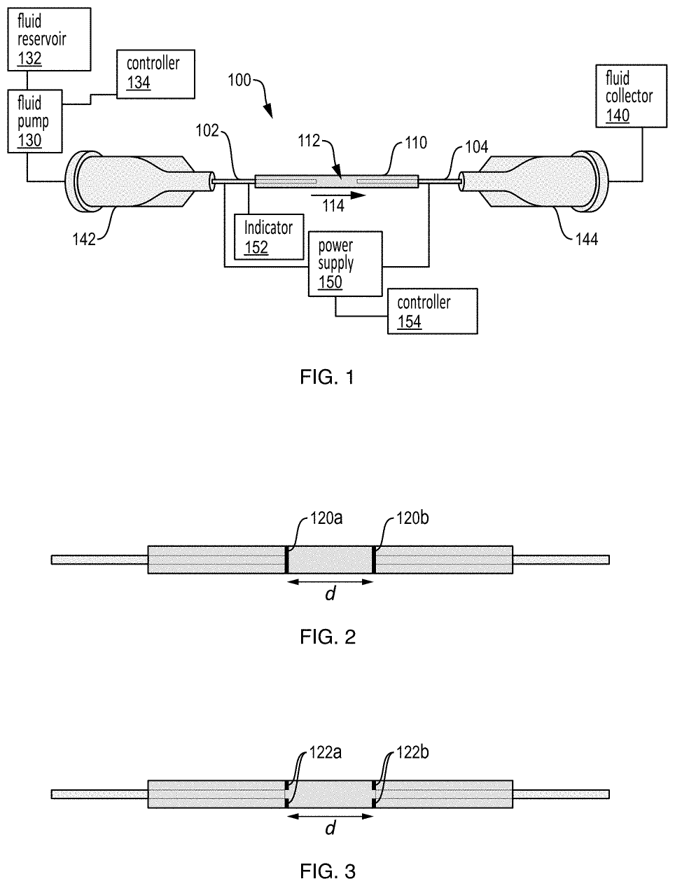

FIG. 1 illustrates an example of an electroporation device 100 in an assembled state that includes conductive elements 102, 104 and an insulating structure 110. The conductive elements are hollow, tubular structures, such as, for example, cannulas comprising a conductive material (e.g., metal cannulas) or syringe needles.

As used herein, the term “tubular structure” or “tube” refers to a hollow structure having a generally elongated body. A tubular structure or tube is generally a cylindrical structure with a cross-section having a circular geometry; however, other cross-sectional geometries are possible (e.g., polygonal, square, triangular, etc.) and are included within the meaning of the terms.

The insulating structure 110 defines a channel 112. The insulating structure 110 can receive and fluidically couple the conductive elements 102, 104. The conductive elements and the channel define a fluid flow path, as indicated by arrow 114, through the device. A fluid media containing cells to be electroporated can thereby travel through the conductive elements and the channel, with electroporation occurring in the channel. The channel can be of a constant diameter and/or can be configured to provide for a constant velocity of fluid flow through the channel.

The insulating structure can also be of a hollow, tubular structure. The insulating structure can be, for example, a polymer tube. The insulating structure can be configured to receive the at least two conductive elements as inserts at opposing ends of the channel 112. As illustrated in FIG. 2, the insulating structure can include markings 120a, 120b indicating an insertion distance of each the at least two conductive elements. The markings can enable a user to assemble the device for use with a particular channel length by placing the conductive elements at a defined distance d from one another. While only one set of markings is shown in FIG. 2 for clarity, more markings can be included. For example, markings can be provided to enable a user to assemble the device for any of several channel lengths (e.g., distances d of 2 mm, 3 mm, 4 mm, etc.).

Alternatively, or in addition, the insulating structure can include stops 122a, 122b for defining an insertion distance for each the at least two conductive elements, as illustrated in FIG. 3. Thus, as a user inserts the conductive elements into the insulating structure, resistance to further insertion is provided by the stops, allowing the conductive elements to be inserted to a defined distance d from one another. While only one set of stops is shown in FIG. 3 for clarity, multiple stops can be included. For example, stops can be included that can provide adequate resistance for providing feedback to a user while permitting further insertion of the conductive elements upon application of additional force.

The conductive elements can be inserted into the channel of the insulating structure with a gap therebetween (i.e., as indicated by distance d) of about 1 mm to about 50 mm, or of about 1 mm to about 10 mm.

The conductive elements can provide for further coupling of the device to a fluid delivery system, a fluid collection system, or both. As illustrated in FIG. 1, the conductive elements 102, 104 are disposed with connecting elements 142, 144. For example, with a conductive element being a metal cannula or a syringe needle, the connecting element can be a syringe hub that couples the connecting element to a syringe or a syringe pump. However, other fluid delivery systems can be connected to the device 100.

As further illustrated in FIG. 1, an upstream conducting element 102 can be coupled, directly or indirectly, to a fluid pump 130. Examples of suitable fluid pumps include syringe pumps, peristaltic pumps, and diaphragm pumps. The fluid pump 130 can be configured to supply fluid from a fluid reservoir 132 to the conductive element 102. For example, the fluid pump 130 can be configured to supply a cell media to the conductive element 102 such that a flow rate of the cell media through the channel is of about 1 mL/min to about 1500 mL/min, or of about 1 mL/min to about 100 mL/min. The fluid pump can be controlled by a controller 134, which can be configured to control the flow rate based upon a selected residence time for cells to be exposed to an electric field in the channel. A downstream conducting element 104 can be coupled, directly or indirectly, to a fluid collector 140 for collection of the cell media following electroporation.

A flow rate to be provided to the channel can be selected based on, in part, a diameter of the channel. As further described in the Exemplification section herein, tubes of three different diameters were tested: 0.5 mm, 0.8 mm, and 1.6 mm. The experimental results demonstrated that electroporation efficiency was generally consistent across the different-diameter devices where fluid velocity was maintained. For example, under a given fluid velocity, a tube with a larger diameter may require a higher total flow rate. A flow rate of 70 mL/min was tested with the use of a 0.8-mm tube, which, when using a 1.6-mm tube, corresponds to a flow rate of 280 mL/min. In a further example, if a tube of 3.2-mm is used, a flow rate of about 1120 mL/min can be used.

A channel having a substantially constant inner diameter can provide for a constant and/or uniform velocity of fluid flowing through the channel, including velocities significantly greater than those typically employed by or achievable with microfluidic devices. For example, a velocity of fluid flowing through the channel, when in configuration with a fluidic pump, can be within range of about 0.1 m/s to about 5 m/s.

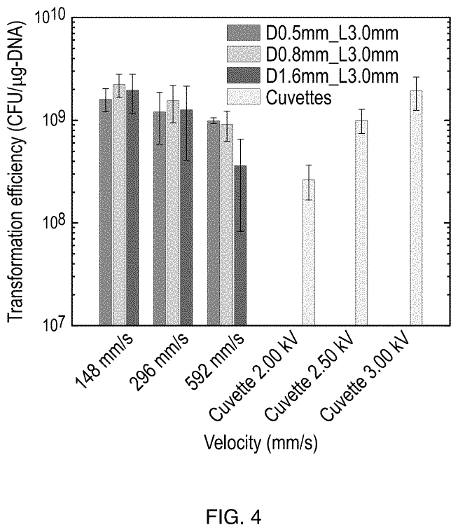

FIG. 4 is a graph illustrating transformation efficiency for the tested tube diameters and demonstrating scalability of the device. A flow rate in the channel can be adjusted accordingly to keep a desired “fluid velocity” unchanged. The experimental results demonstrate that, as long as a given fluid velocity is maintained, electroporation efficiency does not significantly change, regardless of the tube diameter. Thus, the device 100 can be scaled up, provided that a fluidic pump can supply a needed flow rate to maintain the fluidic velocity in the channel.

Referring again to FIG. 1, a power supply 150 can be in operative arrangement with the at least two conductive elements. Examples of suitable power supplies include function generators, amplifiers, DC power supplies, and rechargeable or disposable batteries. A voltage supplied by the power supply can be configured to generate an electric field within the channel of about 0.1 kV/cm to about 100 kV/cm. In some embodiments, a provided electric field strength depends on two factors: distance between the conductive elements (i.e., channel length), and the capability (i.e., output voltage range) of an external power supply. Control of the applied voltage for generating a desired electric field strength in the channel for electroporation can be based on these parameters. The power supply 150 can be in operative arrangement with a controller 154 configured to control an applied voltage based upon a selected electric field strength.

Table 1, below, provides examples of suitable combinations of applied voltages and channel lengths (e.g., gap distance) with resulting field strengths. Generally, field strength=voltage applied/gap.

TABLE 1
Example Field Strengths by Channel Length (Gap) vs. Applied Voltage

| | | Voltage applied | | | | | | | | |
|---|---|---|---|---|---|---|---|---|---|---|
| | | 0.1 kV | 0.5 kV | 1 kV | 1.5 kV | 1.75 kV | 2.00 kV | 2.5 kV | 5.0 kV | 10.0 kV |
| Channel | 0.1 cm | 1.00 | 5.00 | 10.00 | 15.00 | 17.50 | 20.00 | 25.00 | 50.00 | 100.00 |
| gap | 0.2 cm | 0.50 | 2.50 | 5.00 | 7.50 | 8.75 | 10.00 | 12.50 | 25.00 | 50.00 |
| | 0.3 cm | 0.33 | 1.67 | 3.33 | 5.00 | 5.83 | 6.67 | 8.33 | 16.67 | 33.33 |
| | 0.4 cm | 0.25 | 1.25 | 2.50 | 3.75 | 4.38 | 5.00 | 6.25 | 12.50 | 25.00 |
| | 0.5 cm | 0.20 | 1.00 | 2.00 | 3.00 | 3.50 | 4.00 | 5.00 | 10.00 | 20.00 |
| | 0.6 cm | 0.16 | 0.83 | 1.67 | 2.50 | 2.92 | 3.33 | 4.17 | 8.33 | 16.67 |
| | 0.7 cm | 0.14 | 0.71 | 1.43 | 2.14 | 2.50 | 2.86 | 3.57 | 7.14 | 14.29 |
| | 0.8 cm | 0.13 | 0.63 | 1.25 | 1.88 | 2.19 | 2.50 | 3.13 | 6.25 | 12.50 |
| | 0.9 cm | 0.11 | 0.55 | 1.11 | 1.67 | 1.94 | 2.22 | 2.78 | 5.55 | 11.11 |
| | 1.0 cm | 0.10 | 0.50 | 1.00 | 1.50 | 1.75 | 2.00 | 2.50 | 5.00 | 10.0 |

(unit: kV/cm)

As further illustrated in FIG. 1, the device 100 can optionally include an indicator 152 configured to indicate an applied current in the flow path for user feedback.

The conductive elements and the insulating structure can be provided as a unit, or can be assembled and disassembled by a user. One or more parts can be disposable or reusable.

A method of fabricating an electroporation device 100 includes inserting a conductive element at each opposing end of an insulating structure defining a channel. In coupled arrangement, the conductive elements and insulating structure define an electroporation flow path in the channel for flow-through electroporation.

A kit can include a plurality of conductive elements and a plurality of insulating structures for assembly by a user.

Examples of high-throughput electroporation systems that include components such as liquid handling systems and cell-collection units are further shown and described in WO 2017/2103345, the entire teachings of which are incorporated herein by reference. Further description of example devices and test results of prototype assemblies are provided hereinbelow.

Example configurations of high-throughput electroporation devices and systems, and preliminary results obtained with such devices and systems, are further described in the Exemplification section herein.

Taken together, the results established that the disposable, fabrication-free M-TUBE device can process large volumes of bacterial cells with dramatically reduced processing time and effort, without compromising transformation efficiency and cell viability. Due to the simplicity of its fabrication and the wide availability of its components, M-TUBE presents an electroporation strategy that can be immediately implemented in the microbiology community. The flexibility that M-TUBE offers in tuning electroporation conditions such as field strength and residence time make the device a powerful tool for working with hard-to-transform strains. Given the relatively high transformation efficiency compared with cuvettes and its ability to deal with both small and large volumes, M-TUBE has the potential to be a viable alternative to cuvettes and an indispensable tool for applications requiring large volumes such as the creation of mutant libraries.

Example distinguishing features of embodiments of the device may be described as follows:

(1) Fabrication-free preparation: embodiments of the device may be assembled with commercially available disposable syringe needles and plastic tubing and require no microfabrication, mechanical machining, or 3D printing. This allows one to make the device readily by hand-assembling the needles and tubing together, which takes 60˜90 seconds for a single device. Simply by using needles and tubing of varying sizes, one may easily scale up or down the processing volume of the device. The fabrication-free feature allows a microbiologist to implement the device immediately for an application, thereby making the device commercially and academically valuable.

(2) Flow-through based electroporation: the device may perform electroporation by continuously flowing bacteria samples through microfluidic channel(s) (e.g., a microsized plastic tubing) with electrical fields established therein. Compared to the gold standard electroporation cuvette, which perform electroporation in a stop flow with limited processing volume, the device disclosed herein performs electroporation in a continuous, flow-through manner. The continuous, flow-through manner removes a need for extensive manual pipetting; the electroporated bacterial sample can flow directly into recovery media, which potentially improves cell viability and transformation efficiency.

(3) Flexible operation: because the device may simply include commercially-available syringe needles and plastic tubing, the device is compatible with most of commercially-available syringes and, therefore, syringe pumps for sample delivery, both of which are common lab supplies and equipment. Nevertheless, if syringe pumps are not available, one can also consider manually delivering bacterial samples by hand injection with a constant force, which may lead to a desired transformation efficiency given that the device can work at a wide range of flow rates.

(4) Compatible with common electronics equipment: in the device, the electric field is established in the microchannel by applying either an AC or DC signal across the channel. Due to its simplicity, the device is compatible with common laboratory electronics equipment, such as a function generator (AC), DC power supply, high-voltage amplifier, and oscilloscope for the establishment and measurement of electric fields.

(5) Suitable for mass production: the main components of the devices may be disposable syringe needles and plastic tubing, both of which are mass-produced by mature manufacturing processes and are readily available at a very low cost. Because the device may be made simply by assembling syringe needles and plastic tubing together, embodiments of the device may also be mass-produced by existing mass production processes with minor process modification(s). Such suitability for mass production presents a significant commercial value; one example is the gold standard electroporation cuvette.

Example applications with commercial value include the following. Electroporation is used in numerous industries with significant commercial value. By leveraging common lab equipment (e.g., syringe pump, DC power supply, function generator, high-voltage power amplifier), an example embodiment of the device facilitates high volume, automated transfection of both prokaryotic and eukaryotic cells. The technology disclosed herein is of use to any organization involved in the research and development of, for example, novel genetically engineered cells for a variety of applications in synthetic biology, industrial biotechnology, drug discovery, and the human microbiome.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

### EXEMPLIFICATION

### Example 1

### Assembly and Characterization of an M-TUBE Device

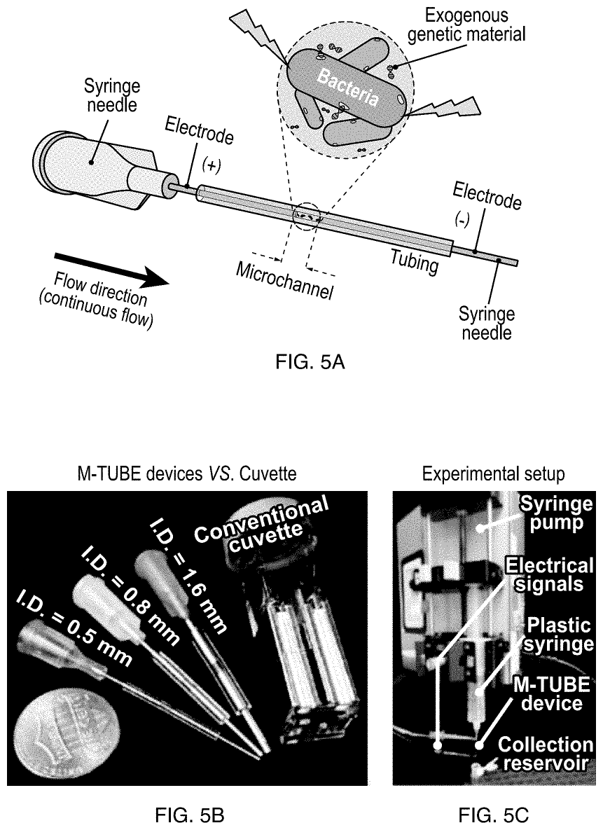

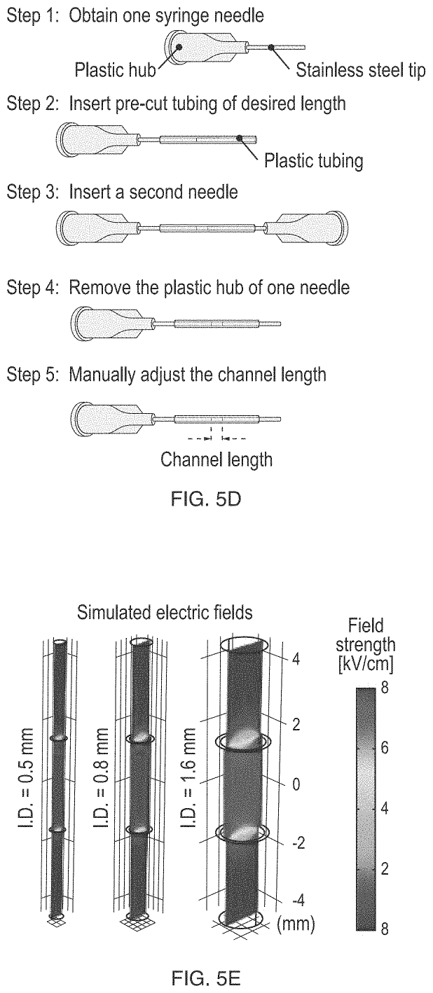

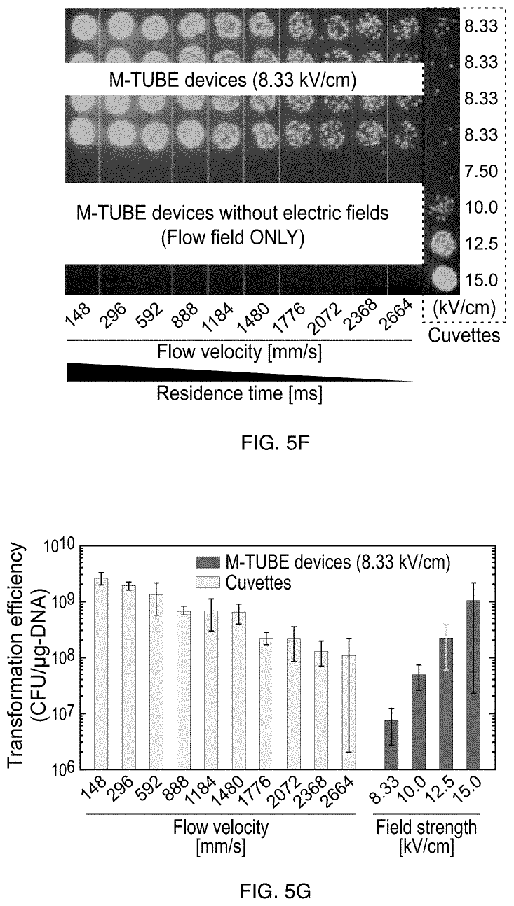

An example M-TUBE device consisted of two syringe needles and one plastic tube of a defined length (FIG. 5A). The plastic tubing served as the microfluidic channel, and the syringe needles served as the two electrodes, which, when connected to an external high-voltage power supply, established an electric field across the tubing microchannel. Upon establishing an electric field in the channel, bacterial cells flowing through the channel can be electrotransformed and uptake surrounding genetic material. The syringe needles and plastic tubing used to assemble the prototype M-TUBE devices are commercially and readily available at low cost (<$0.21 per device), and the overall size of an M-TUBE device is similar to that of a conventional cuvette (FIG. 5B). Because syringe needles of standard common formats can be used, M-TUBE can be attached to any commercially available syringe with complementary connectors and can be conveniently interfaced with any syringe pump for sample delivery (FIG. 5C).

The M-TUBE device can be easily assembled in five steps (FIG. 5D). In brief, device assembly is accomplished by inserting one syringe needle into the plastic tubing cut to a particular length, and a second syringe needle is inserted into the other end of the tubing. Once both needles are inserted, the length of the channel can be manually adjusted to a pre-defined value (Methods) by modifying the gap between the facing ends of the two syringe needles. Assembling a single M-TUBE device required only 90-120 s, far more convenient than typical fabrication processes for microfluidic devices (usually require several days).

Simulations of the electric field established in the tubing microchannel of M-TUBE (FIG. 1E) indicate that the electric field strength is unaffected by the size of the microchannel (i.e., the tubing inner diameter), assuming that the applied voltage (e.g., 2.50 kV) and distance between the two electrodes (gap, or microchannel length) are held constant. This characteristic enables M-TUBE devices to cover a wider range of sample flow rates without having to adjust the applied voltage to maintain the same field strength. The gap of M-TUBE devices can be easily adjusted without additional assembly, unlike devices that rely on microfabrication, CNC machining, or 3D printing [29], providing a simple method for adjusting electric field strength of a device. Another beneficial feature is that the residence time within M-TUBEs can be adjusted to control cell exposure to the electric field. Since M-TUBE electroporates bacterial cells in a continuous flow manner, the residence time is dictated by the fluid velocity (or flow rate), such that residence time decreases with an increase in fluid velocity if the gap is fixed (Table 2). These two features, gap length and flow rate, offer users more flexibility in tuning important electroporation parameters such as the electric field strength and the residence time, respectively, which are not always readily tunable in conventional electroporators.

### Example 2

### Comparison of Cell Viability Between M-TUBE and Conventional Cuvettes

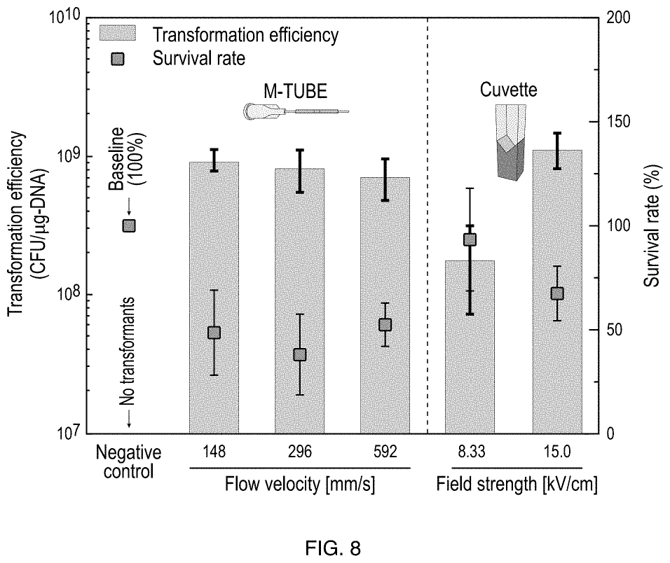

To compare cell viability resulting from use of M-TUBE devices and conventional cuvettes, we conducted three separate rounds of electroporation experiments to compare the cell viability (or the cell survival rate) between using conventional cuvettes and M-TUBE devices. The cell survival rate is defined as the number of viable cells from the electroporated samples to the number of viable cells from the non-electroporated sample. As shown in FIG. 8, M-TUBE devices, compared to cuvette-based electroporation at 8.33 kV/cm, exhibited higher transformation efficiencies across the range of flow rates tested with a compromised cell survival rate of around 50%. On the other hand, M-TUBE devices, when compared to cuvette-based electroporation at 15.0 kV/cm, could achieve a similar efficiency using lower field strengths while maintaining a comparable cell survival rate. Overall, the cell viability from M-TUBE devices seemed slightly lower than those of cuvettes. Nevertheless, we would like to point out that, in this specific series of experiments to characterize the cell viability, electroporation experiments were carried out in the following order (or the order when the samples were resuspended in recovery media): 3 negative-control cuvette electroporations, 3 cuvette electroporations at 8.33 kV/cm, 3 cuvette electroporations at 15.0 kV/cm, 3 M-TUBE electroporations at 148 mm/s, 3 M-TUBE electroporations at 296 mm/s, and then 3 M-TUBE electroporations at 592 mm/s. By the time all the M-TUBE electroporations were finished, electroporated samples from the cuvette groups had been recovering in the recovery medium for over 20 minutes. This may explain why the cell survival rates for M-TUBE devices are slightly lower than those of conventional cuvettes, given the fact that the doubling time of cell growth for most E. coli strains is around 20 minutes.

### Example 3

### Optimization of Bacterial Electroporation with M-TUBE

To establish the utility of M-TUBE, optimize its design, and showcase its ability to electrotransform bacterial cells, we used a strain of E. coli (NEB10β) with high transformation efficiency. The M-TUBE devices employed for most experiments conducted in this study were comprised of a 500-μm diameter tube and 3-mm gap, and were supplied with a voltage of ±2.50 kV or 5.00 kV_{PP} (peak-to-peak AC signal, square wave), which leads to a field strength of 8.33 kV/cm within the microchannel. Cuvettes with 2-mm gaps were used to perform electroporation at different voltages for as a control. We first confirmed that the flow field (or flow shear stress) along the tube does not by itself lead to genetic transformation. In the absence of an electric field, simply flowing cells through M-TUBE at fluid velocities ranging from 148 mm/s (1.8 mL/min) to 2664 mm/s (32.6 mL/min) did not result in any transformation events (FIG. 5F, bottom). By contrast, once a sufficient electric field was established within M-TUBE, colonies were obtained across the entire range of flow rates tested ((FIG. 5F, top), with transformation efficiencies ranging from 10⁸-10¹⁰ CFUs/μg of DNA (FIG. 5G). A reduction in electroporation efficiency was observed as the fluid velocity was increased. This trend was expected because the residence time decreases as the flow rate increases, hence cells are exposed to the electric field for a shorter duration at higher flow rates. Despite the lower efficiency at higher flow rates, the overall efficiency obtained using the M-TUBE device was at least one order of magnitude higher than that obtained using cuvettes with the same field strength (8.33 kV/cm). We also note that, compared to cuvettes (typically used at 10-15 kV/cm), M-TUBE was able to produce a comparable efficiency using a lower electric field. The finding that M-TUBE outperforms cuvettes in terms of transformation efficiency may be due to a synergistic effect of the flow field and the electric field [30].

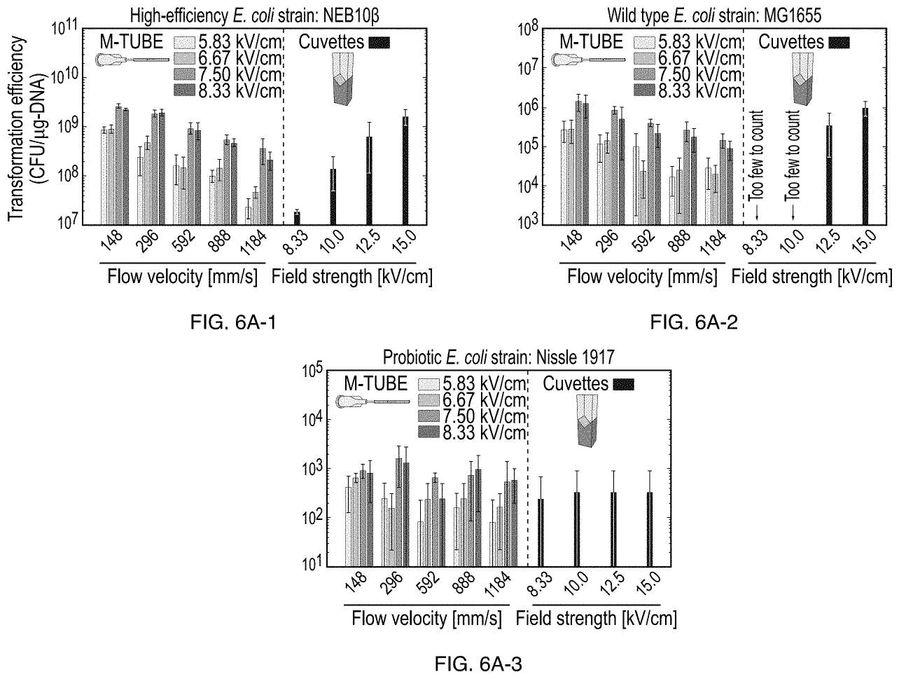

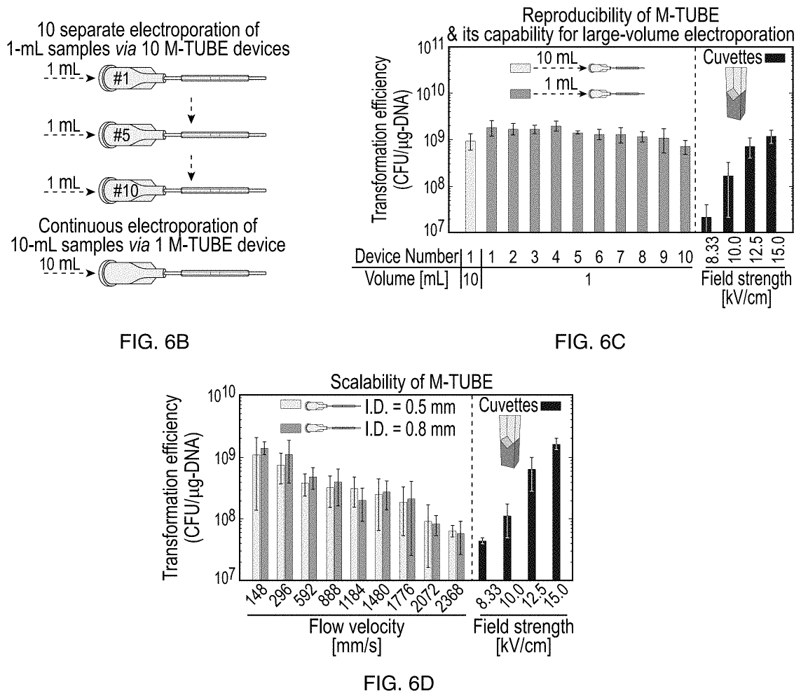

Given the strong dependence of transformation efficiency on field strength in cuvette-based electroporation, we next evaluated how M-TUBE performs across field strengths. Compared to cuvette-based electroporation at 8.33 kV/cm, regardless of the supplied field strength, M-TUBE exhibited higher transformation efficiencies across the range of flow rates tested (FIG. 6A-1). This finding indicates that M-TUBE can either achieve the same efficiency with lower field strengths or higher efficiency with the same field strength. Moreover, electroporation efficiencies with M-TUBE had a smaller standard deviation than those obtained with cuvette-based electroporation. Thus, M-TUBE provides several benefits compared with cuvettes in addition to its high-volume capability.

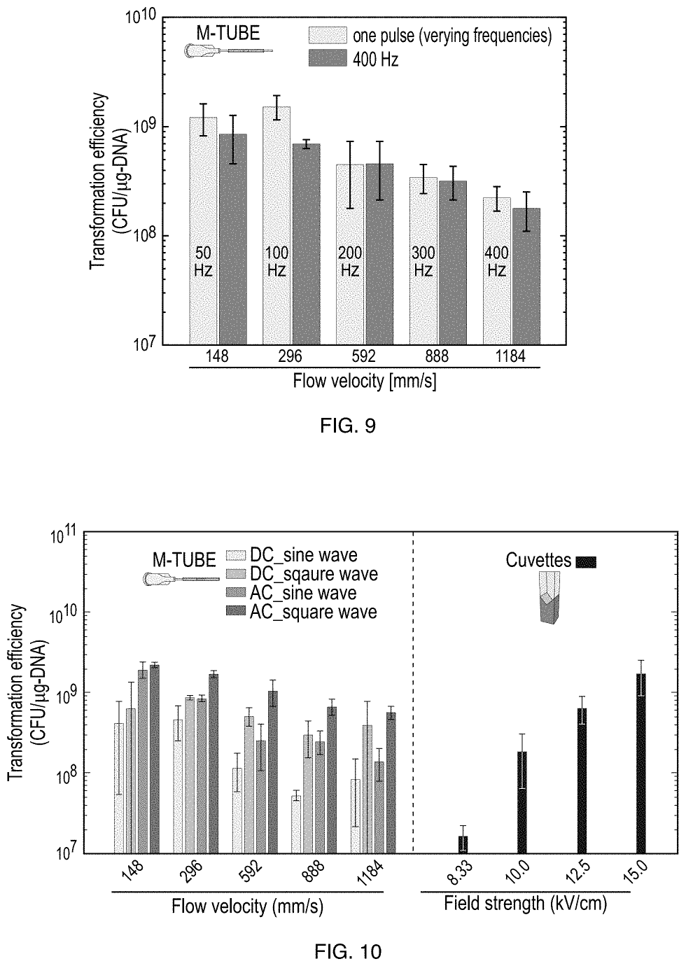

Most M-TUBE electroporation experiments in this study were carried out using an electric field generated with alternating current (AC) rather than direct current (DC). With DC fields, M-TUBE also exhibited higher electroporation efficiency than cuvettes using the same field strength or comparable efficiency using a lower field strength, although efficiency and reproducibility with DC fields were overall lower than with AC fields (FIG. 10). To determine whether M-TUBE transformation efficiency depends on AC field frequency, we conducted electroporation experiments across five fluid velocities in the range of 148 to 1184 mm/s with a distinct frequency (50, 100, 200, 300, 400 Hz) for each fluid velocity so that cells flowing through the microchannel were exposed to only a single pulse (FIG. 9). For a comparison, electroporation was also carried out at a common frequency (400 Hz) for all fluid velocities tested. Electroporation efficiency was largely independent of AC field frequency (FIG. 9). This result contrasted with a previous study that observed frequency dependence [31], potentially due to differences in channel geometry. Regardless, our findings highlight the flexibility of M-TUBE.

M-TUBE exhibits comparable or better efficiency compared with cuvettes across E. coli strains

Motivated by the successful transformation of E. coli NEB10β, M-TUBE was then tested on the wild-type strain E. coli MG1655, which typically has lower transformation efficiency than NEB10β. The results show that M-TUBE maintained higher efficiency than cuvettes for MG1655 (FIG. 6A-2). With a field strength of 8.33 kV/cm, M-TUBE yielded efficiencies at least two orders of magnitude higher than cuvettes; even though cuvettes were supplied with a field strength of 10 kV/cm, the number of successfully transformed colonies was too low to reliably enumerate. To further test M-TUBE performance on E. coli strains, we used M-TUBE to electroporate the probiotic strain Nissle 1917 [27, 28]. While both M-TUBE and cuvettes exhibited much lower electroporation efficiencies for Nissle 1917 compared with MG1655, M-TUBE was comparably efficient to cuvettes and showed slightly better reproducibility (FIG. 6A-3). Moreover, the ability of M-TUBE to process arbitrarily large sample volumes in a continuous fashion means that a desired number of transformed cells of a low-efficiency strain such as Nissle can be obtained with M-TUBE simply by processing a sufficiently large volume. Conversely, using cuvettes for the same goal would be expensive and technically challenging. Overall, M-TUBE showed robust performance across E. coli strains with a wide range of electroporation efficiencies, with performance and reproducibility higher than or comparable to cuvette-based electroporation.

### Example 4

### Assembly has Negligible Effect on Reproducibility of M-TUBE

Since M-TUBE is hand-assembled, small fluctuations in the microchannel length are inevitable across independently assembled M-TUBE devices (even assembled by the same user). Given that the field strength is defined as the ratio of the applied voltage to the microchannel length, we sought to evaluate if the field strength differs significantly across identical but separately assembled M-TUBE devices, thereby causing variation in electroporation performance for NEB10β cells (FIG. 6B, top). We concurrently carried out electroporation of a large-volume sample (10 mL) to demonstrate the capacity of M-TUBE for high-volume electroporation (FIG. 6B, bottom), from which we were able to determine if there is a substantial difference in transformation efficiency between multiple small volume electroporation experiments and continuous flow large volume electroporation. The variation across 10 M-TUBE devices was insignificant and negligible, and each of the tested devices outperformed cuvettes regardless of the field strength (FIG. 6C), confirming that assembly has negligible impact on the reproducibility of the M-TUBE.

Furthermore, M-TUBE was able to electroporate the entire 10-mL sample at a flow rate of 3.6 mL/min with efficiency higher than or comparable to cuvettes (FIG. 6C) and the transformation efficiency for 10 mL of continuous electroporation was not significantly different from that of 10 separate 1-mL experiments. Continuous electroporation of 10 mL is equivalent to 100 individual 0.1-mL cuvette-based electroporations, for which the configuration of M-TUBE that we tested would shorten the entire electroporation time by two to three orders of magnitude (depending on the flow rate). Put in other terms, M-TUBE can process two to three orders of magnitude more volume of sample in a given period of time compared with cuvettes (Table 3). In terms of cost, M-TUBE is at least 10-fold cheaper than cuvettes (Table 4). Moreover, using M-TUBE for large-volume bacterial electroporation can also circumvent the need for manual pipetting by flowing the electroporated sample directly into recovery medium, thereby decreasing total processing time and potentially improving cell viability and transformation efficiency. Taken together, these features make M-TUBE an ideal candidate for large-volume bacterial electroporation.

### Example 5

### M-TUBE Throughput Can be Scaled Up Without Compromising Efficiency

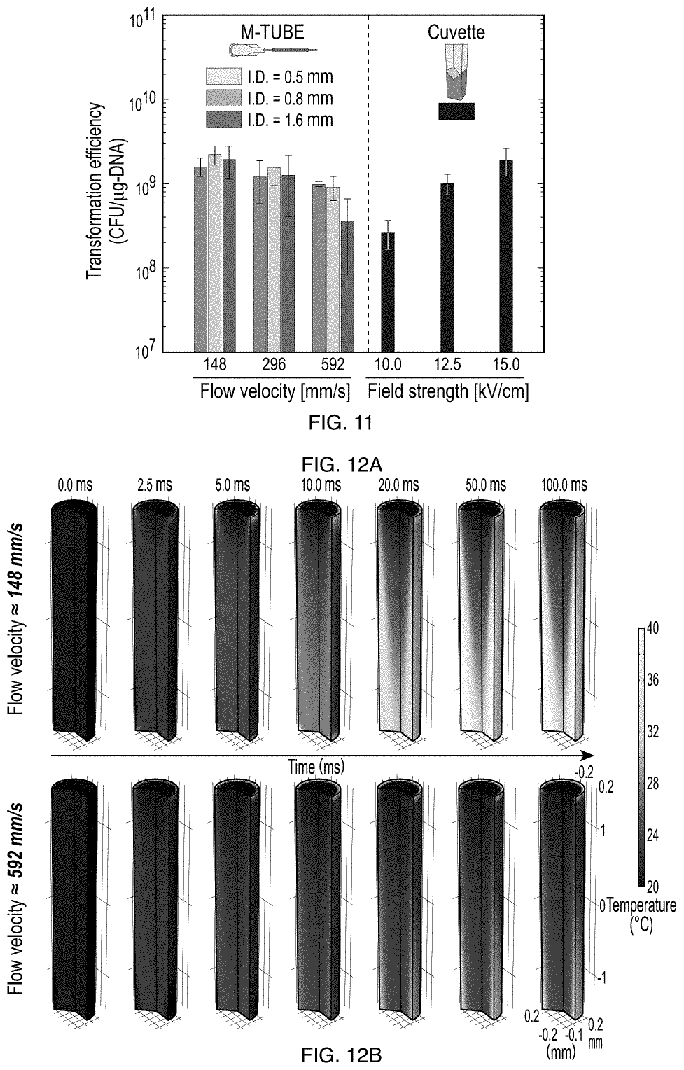

Our next goal was to evaluate the ability to scale up the M-TUBE to process even larger volume samples. To this end, the performance of the M-TUBE device with three different inner diameters was compared (500, 800, and 1600 μm, with the size of syringe needles altered accordingly) (FIG. 6D, FIG. 11). As long as the gap and the fluid velocity were held fixed, M-TUBE devices with different diameters maintained a high electroporation efficiency for NEB10β cells and outperformed cuvettes. With the same fluid velocity, an M-TUBE device with larger diameter would enable processing larger volumes: with a diameter of 1600 μm, an average fluid velocity of 592 mm/s allows for electroporation of ˜70 mL/min, several orders of magnitude more than what is possible with cuvettes. These results again demonstrate the capabilities of M-TUBE for large-volume bacterial electroporation, and confirm that M-TUBE can be readily scaled up without compromising efficiency simply by changing the tubing and syringe needles sizes while maintaining fluid velocity.

### Example 6

### Numerical Evaluation of Joule Heating in M-TUBE Devices

Compared to electroporation of mammalian cells (tens of microns in size), which typically requires electric field strengths <2 kV/cm, successful electroporation of bacterial cells (approximately 1 μm in size) requires field strengths of 10-25 kV/cm. The use of large electric fields introduces the risk of increased Joule heating, which could compromise cell viability. To estimate the magnitude of Joule heating in M-TUBE devices, we conducted numerical modelling of the temperature distribution inside an M-TUBE microchannel under various conditions. For a fluid velocity of 148 mm/s (FIG. 12A), which corresponds to a residence time of approximately 20 ms, simulations predicted a localized temperature increase between approximately 2° C. and approximately 15° C. for an applied electric field of 8.33 kV/cm, dependent on the transient location of cells while flowing through the microchannel.

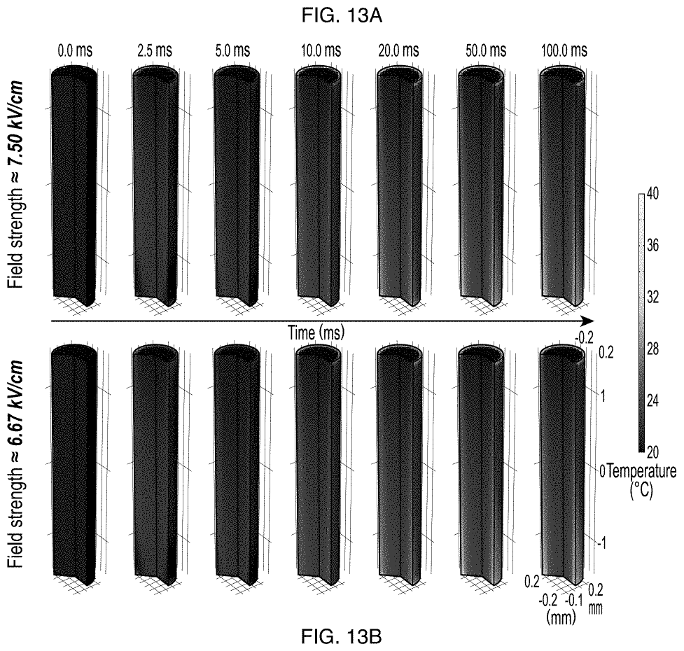

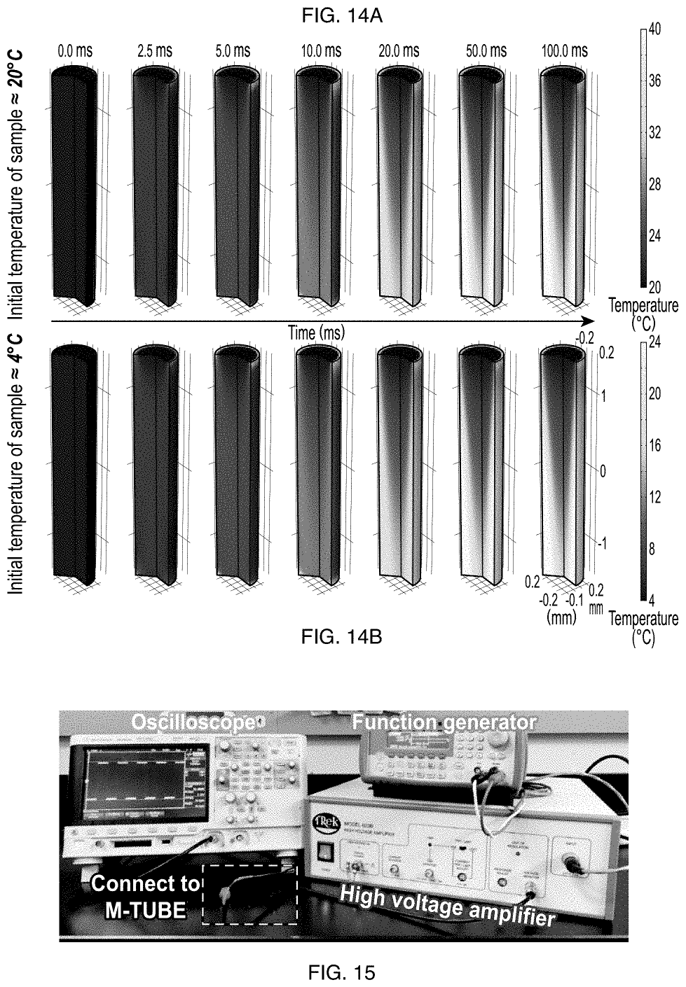

While simulations predicted a maximum temperature increase of up to 15° C., cells would be exposed to these high temperatures for only a short period of time (<20 ms even for the slowest fluid velocities), and simulations predicted that flowing cells at the faster fluid velocity of 592 mm/s, which corresponds to a residence time of approximately 5 ms, would improve heat dissipation by providing better cooling and thereby lower the maximum temperature increase and even out the temperature distribution (FIG. 12B). Application of lower electric fields would also be beneficial for reducing the Joule heating effect (FIGS. 13A, 13B). Moreover, we confirmed numerically that the temperature increase (ΔT) is independent of the initial temperature of the cell sample (FIGS. 14A, 14B). These results suggest that cell samples should be suspended in relatively cold electroporation buffer so that the final temperature inside the channel is below approximately 40° C., beyond which cell viability could be compromised (although many species may be able to survive extremely short periods of heating). Taken together, these simulations indicate that when M-TUBE devices are used to electroporate cells using high-magnitude electric fields, the optimal conditions are higher fluid velocities, buffers with lower conductivities, and cell suspensions in cold buffers. See FIGS. 12A, 12B, 13A, 13B, 14A, 14B.

### Example 7

### Generation of a Transposon Mutant Library in an Anaerobic Gut Commensal with M-TUBE

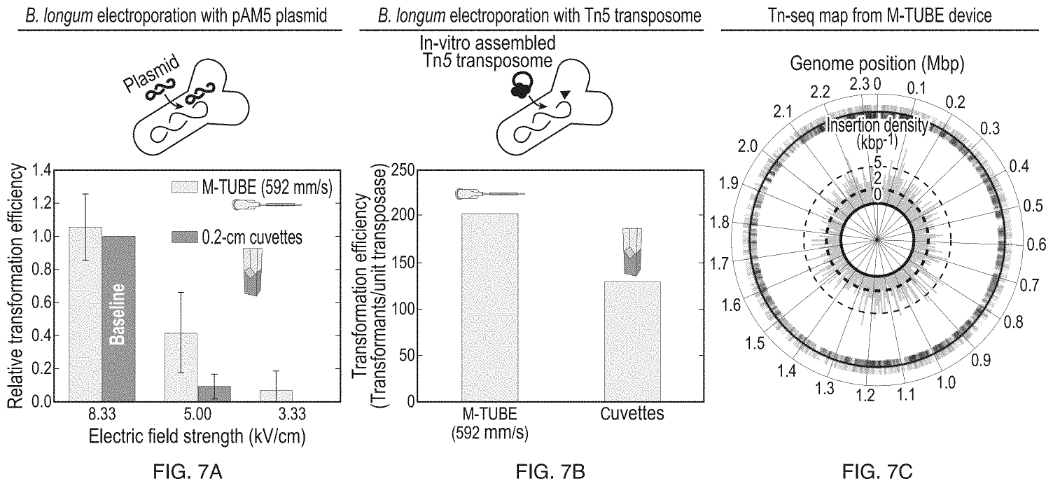

As a demonstration of the utility of M-TUBE in other organisms, we sought to use the system to generate a set of transposon insertion mutants in a human gut commensal. Many of these organisms are obligate anaerobes and hence require more complex handling during growth, washing, and electroporation. We assembled the M-TUBE electroporation platform inside an anerobic chamber and ran an experiment to generate a small-scale transposon insertion pool in Bifidobacterium longum subsp. longum NCIMB8809. B. longum species are used as probiotics and are actively investigated for their health-promoting effects [32]. To identify optimal electroporation conditions for maximizing transposome delivery, we first electroporated B. longum NCIMB8809 cells with the pAM5 plasmid (FIG. 7A, Table 5). As with E. coli, M-TUBE plasmid transformation efficiency was comparable to or higher than that of cuvettes for B. longum (FIG. 7A). With the optimal electroporation conditions, B. longum cells were successfully transformed with in vitro-assembled EZ-Tn5 transposomes, demonstrating its utility both in an anaerobic chamber and for high-throughput transposon mutagenesis (FIGS. 7B, 7C). Like plasmids, M-TUBE transposome electroporation efficiency was comparable to or higher than that of cuvettes. Transposon sequencing of the transformants revealed >2,000 unique transposition events spread across the genome (FIG. 7C). Given these encouraging results, a scaled-up transformation protocol can potentially produce a transposon pool of sufficient diversity for future chemical-genomic investigation using barcode sequencing [33-35]. Furthermore, M-TUBE can have wide applicability for generation of libraries of thousands of transposon mutants, even in bacterial species with complex growth requirements.

### Example 8

### Methods—Materials

Syringe needles of various gauges (16, 20, or 23) with blunt tips were purchased from CML Supply LLC. Plastic tubing of various diameters were purchased from Cole-Parmer: 0.5-mm inner diameter (ID) (PB-0641901), 0.8-mm ID (EW-07407-70), and 1.6-mm ID (EW-07407-71). Plastic syringes of various volumes with Luer-Lok tips were purchased from Thomas Scientific: 30 mL (BD302832), 20 mL (BD302830), and 10 mL (BD302995). Luria broth (LB) (BD244620) and dehydrated agar (BD214010) were purchased from VWR. MRS broth (BD288130) and Reinforced Clostridial Medium (RCM) (CM0149B) were purchased from Fisher Scientific. Carbenicillin disodium salt (C3416), tetracycline (T7660), L-Cysteine (C7352), α-Lactose monohydrate (L2643), and sucrose (S7903) were purchased from Millipore Sigma. Oligonucleotides were purchased from Integrated DNA Technologies.

### Example 9

### Methods—Modelling of the Electric Fields and Temperature Distribution in M-TUBE

To simulate the electric field when using plastic tubing of different diameters to assemble M-TUBE devices and the temperature distribution under different combinations of electroporation conditions, we built a numerical model in COMSOL Multiphysics v6.0 (Burlington, Mass.). The model is based on the multiphysics module of electromagnetic heating, which couples the physics of electric currents, laminar flow, and heat transfer in solids and fluids. To reduce the computational complexity of the model, we used a simplified channel geometry 500 μm in diameter and 3 mm in length that only includes the tips of the two needle electrodes and the microchannel formed between the electrodes. Equations, boundary conditions, assumptions and numerical techniques used to compute electric fields, flow fields and temperatures are similar to previous literatures [19, 36, 37]. To conservatively model the temperature distribution inside an M-TUBE microchannel, we assumed that 10% (v/v) glycerol contributed to the electric conductivity with 0.01 s/m [38-40].

### Example 10

### Methods—Protocol for Preparation of an M-TUBE Device

An M-TUBE device is assembled from two syringe needles and one piece of plastic tubing with a pre-defined length (FIG. 5D). Here, we describe the details of assembly of an M-TUBE device with a microchannel length of 3 mm and a tubing ID of 0.5 mm. First, we cut plastic tubing (50 feet per roll) into 20-mm segments on a cutting mat with metric dimensions. Second, we take two syringe needles of 23 gauge with a tip length of 0.5 in, which has an outer diameter of 0.63 mm that ensures tight fitting between the tubing inner surface and the outer surface of the syringe needle. Next, we insert one of the syringe needles into the tubing and repeatedly rotate back and forth the tubing and/or syringe needle, until the tip of the syringe needle is close to the middle of the tubing and there is also a small portion of the needle for electrical connection that is not inserted into the tubing. We then insert the other syringe needle and rotate back and forth the tubing/syringe needle or the 2nd syringe needle until a gap (i.e., the microchannel length) of a 2-4 mm between the tips of the two syringe needles is established. The gap size can be checked by placing the entire assembly close to a tape measure. After assembling the three components, we remove the plastic hub from either of the syringe needles. Upon removal of the plastic hub, the gap size should then be carefully re-checked with a tape measure, and slight adjustments can be made to establish a gap of 3 mm by gently twisting either needle inward or outward. After this final adjustment, the M-TUBE device is completely assembled.

As discussed above, assembly of one M-TUBE device requires only 60-90 s, hence we typically prepare 50 M-TUBE devices at a time, in ˜1 h. The M-TUBE devices are placed in a Petri dish, which is sterilized in a biosafety cabinet with UV irradiation overnight. After UV sterilization, M-TUBE devices are stored in a −20° C. freezer or refrigerator until just before conducting electroporation experiments, a step similar to the pre-chilling of electroporation cuvettes.

To prepare M-TUBE devices with other tubing sizes, all steps remain unchanged, with the plastic tubing and syringe needles having complementary outer diameters in their tips.

### Example 11

### Methods—The External High-Voltage Power Supply System

The external high-voltage power supply (FIG. 15) included of a function generator (Agilent Technologies, 33220A), a high-voltage amplifier (Trek Inc., 623B), and an oscilloscope (Agilent Technologies, DSO-X 2022A). The function generator supplies preprogrammed electric signals (AC or DC, sine or square waves, frequency, voltage, etc) to the high-voltage amplifier, which amplifies the signals by 1,000 fold. The oscilloscope monitors the amplified signals to ensure the correct output. The function generator provides non-amplified signals to the amplifier through a BNC cable, and the amplifier outputs the amplified signals through a pair of high-voltage cables, which were customized with alligator clips or test clips and connected to the two electrodes of an M-TUBE device. On/off switching of the high-voltage signals was primarily controlled by engaging and disengaging a trigger button on the function generator. The function generator, amplifier and oscilloscope used in this study are standard electronic equipment that can be accessed in many research laboratories/facilities or readily acquired.

### Example 12

### Methods—Culturing and Preparation of E. coli Strains

Three E. coli strains, including NEB10β (New England Biolabs), K-12 MG1655 (Coli Genetic Stock Center, Yale University) and Nissle 1917 (Mutaflor®, Canada), were employed in this study to test the M-TUBE device. The strains, unless otherwise specified, were cultured, harvested, and made electrocompetent using the same conditions. In brief, glycerol stocks were inoculated into two 14-mL cultures tubes containing 6 mL of LB medium and incubated at 37° C. and 250 rpm. The next morning, 5 mL from each overnight culture was inoculated into 245 mL of LB and grown at 37° C. and 200 rpm to an OD600 of 0.5-0.7. Note that each set of E. coli experiments involved 15-20 mL of electrocompetent cells at OD600=10, which required two 250-mL cultures. Each 250 mL culture was divided equally into six 50-mL centrifuge tubes and spun down at 4° C. and 3500 rpm for 10 min using an Allegra 64R centrifuge (Beckman Coulter). The supernatant was discarded and 6 mL of ice-cold 10% glycerol was used to wash and combine the six cell pellets into one suspension. Each 6-mL cell suspension was equally divided into four 2.0-mL microcentrifuge tubes. The eight microcentrifuge tubes generated from the two 250-mL cultures were centrifuged at 4° C. and 8000 rpm for 5 min, the supernatants were discarded, and 1 mL of ice-cold 10% glycerol was used to wash and resuspend the pellet in each of the eight tubes. These washing steps were repeated twice more. Next, all cell pellets were combined into a concentrated suspension using 8 mL of ice-cold 10% glycerol and the cell concentration (typically OD600=20-30) was measured using a UV spectrophotometer (UV-1800, Shimadzu). Depending on the measured concentration, a final sample with OD600=10 was prepared by adding an appropriate volume of ice-cold 10% glycerol. This sample was placed on ice prior to electroporation. DNA plasmids (Parts Registry K176011) [19] encoding ampicillin resistance and green fluorescent protein (GFP) were added to this sample at a final concentration of 0.1 ng/μL for NEB10β and MG1655 cultures; for Nissle 1917, a final concentration of 1 ng/μL was employed so that the number of colony forming units (CFUs) was above the limit of detection. For electroporation, the sample was loaded into a 30-mL plastic syringe (see section on M-TUBE operation).

### Example 13

### Methods—B. longum Culturing and Preparation for M-TUBE Electroporation with Plasmid DNA

A 5-mL B. longum culture was maintained in an anaerobic chamber (Coy) via daily dilution into fresh medium to prepare for electroporation. Briefly, 1 mL of a B. longum culture was inoculated into 9 mL of MRS medium in a culture tube, and five additional serially diluted (at 1:10 ratio) cultures were prepared; these six cultures were incubated at 37° C. overnight. The next morning, the optical density of each culture was measured using a spectrometer, and the culture with OD600=3-4 was used for subsequent outgrowth. The selected culture was diluted to OD600=0.54 in 60-70 mL and grown to OD600=1.5-2, after which cells were harvested and made electrocompetent following the same steps described above for E. coli. The 60-70 mL were then divided equally into two 50-mL centrifuge tubes and spun down outside the anaerobic chamber at 4° C. and 3500 rpm for 10 min using an Allegra 64R ultracentrifuge (Beckman Coulter). Next, the two 50-mL centrifuge tubes were returned to the anaerobic chamber, the supernatant was discarded, and 5 mL of ice-cold 10% glycerol were used to wash and combine the two cell pellets into one suspension. The 5-mL cell suspension was divided equally into four 2-mL microcentrifuge tubes. The four tubes were centrifuged inside the chamber at room temperature and 10,000 rpm for 2 min using an Eppendorf 5418 microcentrifuge, the supernatants were discarded, and 1 mL of ice-cold 10% glycerol was used to wash and resuspend the pellet in each of the 4 tubes. These washing steps were repeated two more times. Next, all pellets were combined into a concentrated suspension using 5 mL of ice-cold 10% glycerol. Depending on the concentration, the final sample at OD600=10 was prepared by adding the appropriate volume of ice-cold 10% glycerol and then placed on ice prior to electroporation. The pAMS plasmid encoding tetracycline resistance was added to the sample at a final concentration of 2 ng/μL. The mixture of the plasmid DNA with the cells was loaded into a 10-mL plastic syringe for electroporation.

### Example 14

### Methods—Transposon Mutagenesis of B. longum NCIMB8809

Previous transformation protocols [41-43] were combined with minor modifications to prepare electrocompetent cells of B. longum NCIMB8809. Briefly, a glycerol stock of B. longum NCIMB8809 was recovered for 24 h in 5 mL of MRS broth (MRS media, Difco) at 37° C. and passaged overnight (16 h) in 10 mL of MRS in a 10-fold dilution series. The next morning, the incubator temperature was raised to 40° C. and one of the overnight cultures in the dilution series was used inoculate 50 mL of MRS (MRS media, HIMEDIA) in a 250-mL Erlenmeyer flask at an initial OD600 (optical density at λ=600 nm) of 0.18, as measured by a 96-well plate reader (Epoch2, BioTek) in a 96-well flat bottom microplate (Grenier Bio-One, Cat. #655161) with 200 μL of culture per well. In the dilution series, the overnight culture with the lowest optical density that still provided enough cells to proceed was used to inoculate the next culture. The 50 mL of culture in HIMEDIA-brand MRS was grown to an OD600 of 1.0 and used to inoculate MRS broth reconstituted from individual components, modified with 1% lactose as the sole carbon source and an additional 133 mM NaCl, at an initial OD600 of 0.18. This culture was harvested at an OD600 of 0.5, pelleted, washed three times with 15% glycerol, and resuspended at an OD600 of 6.7 in 15% (v/v) glycerol. To harvest the cells, the culture was moved to a pre-reduced 50 mL conical tube (Fisher Scientific, Cat. #06-443-19) on ice, brought out of the anaerobic chamber, centrifuged for 10 min at 3,428g (Centrifuge 5920R, Eppendorf), and transferred back into the anaerobic chamber. After cells were harvested, the incubator temperature was lowered back down to 37° C. Subsequent washes were performed at a volume of 5 mL in 5-mL Eppendorf tubes (Cat. #0030122321, Eppendorf) and pelleted with a compatible microcentrifuge (MC-24™ Touch, Benchmark Scientific) that had been brought into the chamber, using 2-min 10,000 g centrifugation steps. Transposomes were assembled in vitro by mixing an erythromycin resistance cassette with commercially available EZ-Tn5 transposase according to manufacturer's instructions. Transposomes were mixed with competent cells at a concentration of 2U transposase/mL competent cells and electroporated using the M-TUBE device (see below). Electroporated cells were recovered for 2 h at 37° C., concentrated 10-fold through centrifugation and resuspension in MRS, and plated on RCM-agar plates with 5 μg/mL erythromycin. Colonies were harvested for sequencing after —36 h of growth at 37° C.

### Example 15

### Methods—Electroporation of E. coli Strains Using M-TUBE

The final sample of cells mixed with plasmid DNA was loaded into a plastic syringe, which was mounted on a syringe pump (Legato 210P, KD Scientific) that could be operated horizontally or vertically. To prevent bending of the plastic tubing of the M-TUBE device and to enable convenient collection of the electroporated sample directly into tubes, we typically operate the syringe pump as shown in Fig. lc. After arranging the pump to operate vertically, an M-TUBE device was attached to the sample-loaded syringe via Luer-Lok connection, and the two syringe-needle electrodes were connected to the external high-voltage power supply system (FIG. 15), which consists of a function generator, a high-voltage amplifier, and an oscilloscope (Agilent Technologies, DSO-X 2022A). Upon confirming a tight connection between the M-TUBE device and the power supply, we pre-filled the M-TUBE microchannel by infusing the cell sample at a relatively low flow rate (typically 250-500 μL/min), to prevent air bubbles and thereby arcing/sparking in M-TUBE, until we visually confirmed that the microchannel was filled with the liquid cell sample. Next, a collection tube (reservoir) was placed underneath the M-TUBE device (FIG. 5C) so that the electroporated sample could be directly and automatically collected. We programmed the pumping parameters including target pumping volume and pumping flow rate, and started flow using the syringe pump at the pre-set flow rate; immediately after starting flow, we started the application of electric signals to the M-TUBE device to initiate electroporation.

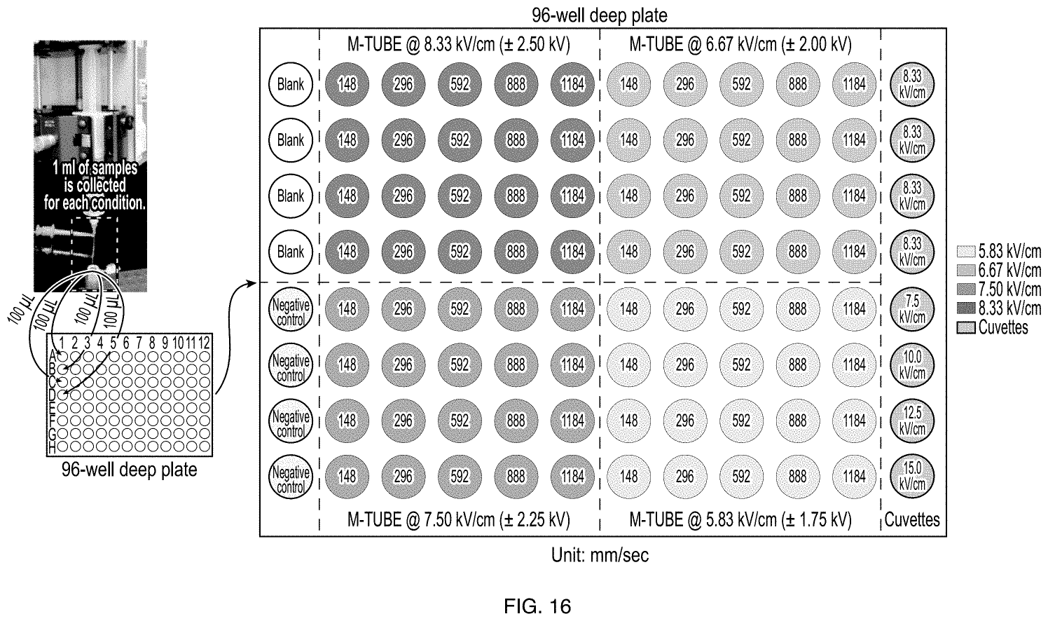

As a positive control, the same batch of electrocompetent cells was also electroporated at various field strengths using 0.2-cm electroporation cuvettes (VWR, 89047-208). One hundred microliters were pipetted into a pre-chilled electroporation cuvette. Each cuvette was pulsed with an electroporator (MicroPulser™, Bio-Rad) at field strengths including 8.33 kV/cm, 10.0 kV/cm, 12.5 kV/cm, and 15 kV/cm with time constants between 5.0-5.5 ms. Immediately after the application of electric pulses to each cuvette, 900 μL of pre-warmed (˜37° C.) LB recovery medium were added to each cuvette, and the 100-μL electroporated cells was mixed with the 900-μL recovery medium via pipetting. We then aspirated as much electroporate sample volume as possible from the cuvette and dispensed it into designated wells on a 96-well deep plate (FIG. 16), along with the electroporated samples from M-TUBE for subsequent recovery at 37° C. for 1 h.

### Example 16

### Methods—Electroporation of B. longum via M-TUBE

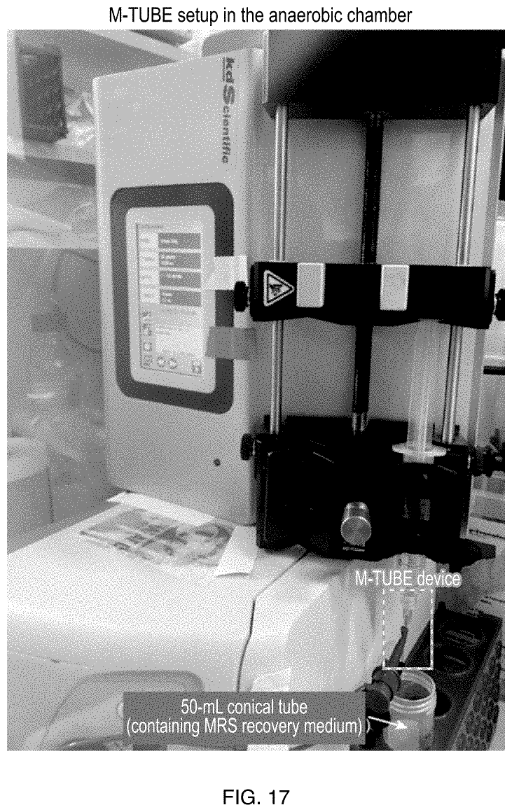

Most steps for B. longum were the same as for E. coli described above; the differences are described here. After pre-filling an M-TUBE device with the B. longum sample, a 50-mL conical tube (reservoir) containing MRS recovery medium was placed underneath the M-TUBE device (FIG. 17), so that electroporated B. longum cells could be directly and automatically flowed into the recovery medium. For B. longum electroporation with M-TUBE, one flow rate (7.2 mL/min, or 592 mm/s for the 0.5-mm M-TUBE device) was tested at three field strengths (3.33, 5.00, and 8.33 kV/cm).

As a positive control, the same batch of electrocompetent cells was electroporated at the same three field strengths using 0.2-cm electroporation cuvettes. One hundred microliters of the final cell sample were pipetted into a pre-chilled electroporation cuvette. Each cuvette was pulsed by the electroporator with time constants ranging between 5.4-5.8 ms. Immediately after the application of an electric pulse, 1000 μL of pre-warmed (˜37° C.) LB recovery medium were added to each cuvette and mixed with the cells via pipetting. We then aspirated as much electroporated sample volume as possible from the cuvette and dispensed it into a 1.5-mL microcentrifuge tube.

### Example 17

### Methods—Collection, Recovery, and Evaluation of Electroporated E. coli Samples

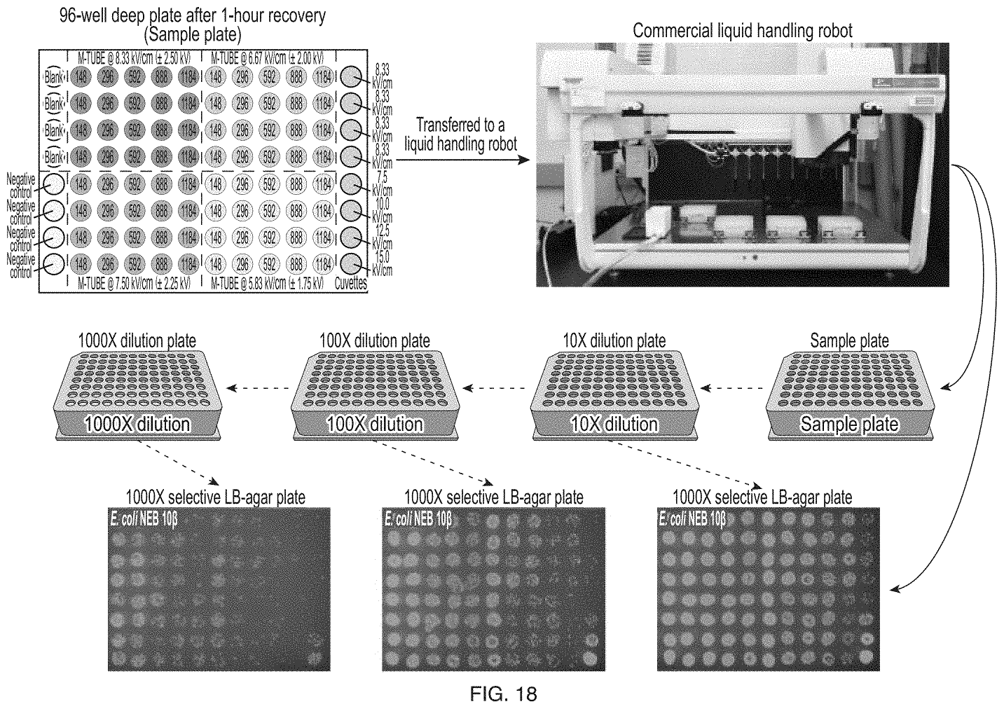

In each set of E. coli experiments, a range of flow rates and electric field strengths were tested; for each combination of testing conditions, 1 mL of electroporated sample was collected in a microcentrifuge tube. One hundred microliters of the electroporated sample was aspirated and dispensed into each of four wells of a 96 deep-well plate containing LB recovery medium (FIG. 16). In each 96-well plate, we were able to test 20 combinations of electroporation conditions. After filling all designated wells of the 96-well plate, the plate was incubated in a shaking incubator at 37° C. and 250 rpm for 1 h. After 1 h of recovery, the 96-well sample plate was placed in a designated position on a liquid handling robot (Janus BioTx Pro Plus, PerkinElmer) for automated serial dilution (FIG. 18): 10×, 100× and 1000× dilution for E. coli NEB10β; 10× and 100× dilution for E. coli K12 MG1655 or Nissle 1917. Following serial dilution, 5 μL from each well were dispensed onto LB-agar plates containing 50 μg/mL carbenicillin, and the selective plates were incubated overnight at 37° C. The next morning, each plate was photographed for CFU counting.

### Example 18

### Methods—Collection, Recovery, and Evaluation of Electroporated B. longum Samples

After electroporating B. longum using M-TUBE, 1 mL of cells was flowed directly into 10 mL of MRS recovery medium. B. longum samples electroporated by M-TUBE or in cuvettes were incubated at 37° C. for 3 h. Following recovery, 1.1 mL from each M-TUBE or cuvette sample were aspirated and pipetted into separate 1.5-mL microcentrifuge tubes and spun down at 10,000 rpm for 2 min. The supernatants were discarded and 200 μL of MRS medium were added into each 1.5-mL tubes to resuspend the cell pellets. Next, the 200-μL suspension was plated onto RCM-agar plates with 10 μg/mL tetracycline, and the selective plates were incubated at 37° C. for at least 48 h. Following the 48-h incubation, each plate was photographed for CFU counting.

### Example 19

### Methods—CFU Quantification

Photos of selective plates for electroporation with plasmids were captured using an iPhone 11 (Apple) on a tripod with a remote shutter. The photos were imported to ImageJ (NIH) and CFU.Ai v. 1.1 for enumerating the CFUs. The transformation efficiency was defined as the number of CFUs on selective plates per μg of DNA.

### Example 20

### Methods—Preparing a Tn-seq Library for B. longum NCIMB8809

Erythromycin-resistant colonies from the Tn5 transposome electroporation were scraped from the selective plates into 500 μL of MRS broth (MRS media, Difco) for each Petri dish. Samples from this suspension were taken, glycerol (Fisher Bioreagents, Cat. #BP229-1) was added to a final concentration of 15% (v/v), and the cryostocks were stored in 11-mm crimp vials (Thermo Scientific™, Cat. #C4011-11) with sealed aluminum crimp caps (Thermo Scientific™, Cat. #11-03-400) at −80° C. Simultaneously, most of the suspension was stored directly at −20° C. for subsequent DNA isolation. Genomic DNA (gDNA) was isolated from the colony suspension using a DNeasy Blood and Tissue Kit (QIAGEN, Cat. #69506) according to the manufacturer's protocol for Gram-positive organisms.

Isolated gDNA was first sheared in a Covaris S220 Sonicator with microTUBE AFA fiber pre-slit snap-cap tubes (Covaris, Cat. #520045) according to the manufacturer's instructions for 300-bp fragments. A KAPA HyperPrep Kit (Roche, 07962312001) with custom oligos was then used to prepare the library. Briefly, sonicated gDNA was subjected to a dual bead-based size selection using AMPure XP magnetic beads (Beckman Coulter, Cat. #A63881) according to the manufacturer's instructions for 300-bp sized fragments. An end-repair and A-tailing reaction was performed followed by an adaptor ligation by following the KAPA HyperPrep protocol and using a custom adaptor (Table 5). After a one-sided bead cleanup, the entire sample of adaptor-ligated gDNA fragments was used as input for a PCR reaction that simultaneously amplified transposon-gDNA junctions and added Illumina TruSeq adaptors. An Ultra II Q5 PCR mix (New England Biolabs, Cat. #E7649A) was used for all PCR reaction components except the template DNA and custom primers (Table 5). The PCR reaction involved an initial denaturation step of 98° C. for 2 min, followed by 25 cycles of three steps: 98° C. for 30 s, 65° C. for 20 s, and 72° C. for 30 s. After a final extension at 72° C. for 10 min, the sample was cleaned up using a NucleoSpin® Gel and PCR Clean-up kit (Machery-Nagel, Cat. #740609.250). The Tn-seq library was run on a MiSeq (Illumina, Cat. #SY-410-1003), with the 150-cycle MiSeq Reagent Kit V3 (MS 3001), 150-bp read 1 length, and no indexing reads.

### Example 21

### Diagrams and Results

FIGS. 5A-G are diagrams related to an embodiment of an M-TUBE, which is a fabrication-free, microfluidics tubing-based bacterial electroporation device that is simple to assemble and exhibits higher electroporation efficiency than cuvettes.

FIG. 5A is a schematic diagram of an embodiment of the M-TUBE device. As illustrated, the device includes two syringe needles and one piece of plastic tubing of pre-defined length. The two syringe needles and plastic tubing serve as the two electrodes and microchannel, respectively. When the two electrodes are connected to an external power supply (or electrical signal generator), an electric field is established within the microchannel, where bacterial electroporation can take place.

FIG. 5B is a photograph of M-TUBE devices with three inner diameters (I.D.) are all similar in size to a conventional cuvette.

FIG. 5C is a photograph of the experiment setup when using the M-TUBE device. Since the M-TUBE device is made from standard, commercially available syringe needles and plastic tubing, it can be readily attached to syringe pumps for automated sample delivery, removing the need for manually pipetting samples.

FIG. 5D is a diagram of a detailed breakdown of the protocol for M-TUBE assembly. One device can be completely assembled in 90-120 s. The total cost of parts is currently less than $0.22 USD, and this price could be lowered if parts are bought in bulk.

FIG. 5E is a diagram of simulations of the electric field established in M-TUBE devices using COMSOL Multiphysics 5.5 predict similar field strengths irrespective of I.D.

FIG. 5F is a diagram of a spot-dilution assay to quantify viability on selective plates when E. coli NEB1013 cells were flowed through the device with a plasmid encoding ampicillin resistance and GFP (Table 5) in the presence or absence of an electric field. Transformation was dependent on the electric field. For M-TUBE devices, a voltage of ±2.50 kV (AC field) was applied, which results in an electric field of 8.33 kV/cm. The same batch of cells was used to conduct cuvette-based electroporation as a comparison.

FIG. 5G is a diagram that provides a comparison of transformation efficiency (colony forming units (CFUs) per μg of DNA) corresponding to the plates in FIG. 5F. The electroporation efficiency of M-TUBE decreased as the fluid velocity was increased, as expected due to the shorter duration of exposure to the electric field. Regardless of the fluid velocity, the efficiency of M-TUBE was at least one order of magnitude higher than that of cuvettes with the same field strength (8.33 kV/cm). Data represent the average (n≥3) and error bars represent 1 standard deviation. The data underlying FIGS. 5E and 5G can be found, respectively, in FIG. 8 Data and FIG. 9 data.

FIGS. 6A-1,2,3 through 6C are diagrams showing that an embodiment of the M-TUBE device exhibits higher efficiency than cuvettes across E. coli strains, is reproducible, and maintains high efficiency across tubing sizes.

FIGS. 6A-1, -2, and -3 provide a comparison of M-TUBE device performance when transforming the high-efficiency strain NEB1013, the wild-type strain MG1655, and the probiotic strain Nissle 1917 across voltages and fluid velocities. M-TUBE outperformed cuvettes at an equivalent electric field strength for all strains. Data represent the average (n≥3) and error bars represent 1 standard deviation.

FIG. 6B is a schematic diagram of an experiment comparing 10 separate 1 mL electroporations and 1 continuous electroporation of a 10 mL sample.

FIG. 6C is a plot showing transformation efficiency for the experiments in FIG. 6B that demonstrates that sample volume can be increased without compromising efficiency. Data represent the average (n≥3) and error bars represent 1 standard deviation. The same batch of cells was used to conduct cuvette-based electroporation as a comparison.

FIG. 6D is a plot showing that transformation efficiency was similar across 0.5-mm and 0.8-mm diameter M-TUBE devices. For M-TUBE devices, a voltage of ±2.50 kV (AC field) was applied, which results in an electric field of 8.33 kV/cm. Data represent the average (n≥3) and error bars represent 1 standard deviation. The data underlying this figure can be found in FIG. 9 data.

FIGS. 7A-7C are diagrams that show that M-TUBE efficiently transforms anaerobic bacteria and enables transposon insertion mutagenesis.

In particular, FIG. 7A is a plot that shows a comparison of M-TUBE performance during electrotransformation of B. longum NCIMB8809 with the plasmid pAMS at various electric field strengths. For M-TUBE devices, voltages of ±2.50, ±1.50, and ±1.00 kV (AC field) were applied to produce electric fields of 8.33, 5.00, and 3.33 kV/cm, respectively. A fluid velocity of 592 mm/s was used for the M-TUBE device because the —5 ms residence time with an M-TUBE inner diameter of 0.5 mm is similar to the time constant observed in cuvette electroporation (5.2-5.6 ms). Data represent the average (n≥3) and error bars represent 1 standard deviation.

FIG. 7B is a plot that shows a comparison of M-TUBE performance during electrotransformation of B. longum NCIMB8809 with Tn5 transposome. For the M-TUBE device, a field strength of 8.33 kV/cm and fluid velocity of 592 mm/s were used, motivated by the results in FIG. 7A.

FIG. 7C is a plot that shows transposon insertions recovered from Tn5 transposome electroporation are spread approximately uniformly across the B. longum NCIMB8809 genome. The locations of individual mapped insertions are recorded on the outer circle. Green ticks on the outside indicate insertions in the positive (+) orientation, blue ticks on the inside indicate insertions in the negative (−) orientation. The insertion density (kbp-1) (both positive and negative orientation) is plotted in 1-kbp bins on the inner circle. Transposon insertions are distributed throughout the genome in both the positive and negative orientations, indicating that B. longum NCIMB8809 can be transformed by Tn5 transposomes using M-TUBE without major insertional bias.

FIG. 8 is a plot that shows a comparison of transformation efficiency and cell viability between when using M-TUBE devices and conventional cuvettes. M-TUBE devices, when compared to cuvette-based electroporation at 15.0 kV/cm, could achieve a similar efficiency using lower field strengths while maintaining a comparable cell survival rate. For M-TUBE devices, a voltage of ±2.50 kV (AC field) was applied, which results in an electric field of 8.33 kV/cm. Data represent the average (n≥3) and error bars represent 1 standard deviation.

FIG. 9 is a plot that shows dependence of M-TUBE's electroporation efficiency on the frequency of AC fields applied. The results reveal that in M-TUBE devices, the electroporation efficiency is independent of the frequency of AC fields applied. For M-TUBE devices, a voltage of ±2.50 kV (AC field) was applied, which results in an electric field of 8.33 kV/cm. Data represent the average (n≥3) and error bars represent 1 standard deviation.

FIG. 10 is a plot that shows a comparison of M-TUBE device performance when the device is supplied with alternating current (AC) fields and direct current (DC) fields. With DC fields, M-TUBE device can also perform electroporation with a higher efficiency using the same field strength or a comparable efficiency using a lower field strength, when compared to cuvettes. Overall, the electroporation efficiency and reproducibility when using DC fields are lower than those obtained when using AC fields. For M-TUBE devices, a voltage of ±2.50 kV (AC field) or 0˜2.50 kV (DC fields with a duty cycle of 95%) was applied, which results in an electric field of 8.33 kV/cm. Data represent the average (n≥3) and error bars represent 1 standard deviation.

FIG. 11 is a plot that shows that transformation efficiency is maintained across M-TUBE devices with different diameters. To evaluate the scalability of M-TUBE further, M-TUBE devices made using plastic tubing with 0.5-mm, 0.8-mm and 1.6-mm inner diameters and compared to conventional cuvettes. A voltage of ±2.50 kV (AC field) was applied to each M-TUBE device, resulting in an electric field of 8.33 kV/cm. The same batch of cells was used to conduct electroporation with 0.2-mm cuvettes and various voltages as a comparison. Data represent the average (n≥3) and error bars represent 1 standard deviation.

FIGS. 12A, B are diagrams of simulated temperature distribution in the microchannel of M-TUBE when cell samples are flowed through the microchannel at the fluid velocity of FIG. 12A) 148 mm/s and FIG. 12B) 592 mm/s. Higher flow rates could be beneficial to improve heat dissipation, thereby reducing localized increase of temperature. The geometry of M-TUBE used for simulation is 500 μm in diameter and 3 mm in length, and a voltage of 2.50 kV is applied, which leads to an electric field of 8.33 kV/cm. The initial temperatures of cell sample are set to 20° C. Data represent the average (n≥3) and error bars represent 1 standard deviation.

FIGS. 13A, B are diagrams of simulated temperature distribution in the microchannel of M-TUBE when a voltage of FIG. 13A) 2.25 kV (or 7.50 kV/cm) or FIG. 13B) 2.00 kV (or 6.67 kV/cm) is applied. As the field strength is decreased, the localized increase in temperature is reduced. The geometry of M-TUBE used for simulation is 500 μm in diameter and 3 mm in length. Cell samples are flowed through the microchannel at the same fluid velocity of 592 mm/s. The initial temperatures of cell sample are set to 20° C.

FIGS. 14A, B are diagrams of simulated temperature distribution in the microchannel of M-TUBE when cell samples with an initial temperature of FIG. 14A) 20° C. or FIG. 14B) 4° C. are flowed through the channel. When the same fluid velocity is employed, the temperature increase (ΔT) are identical regardless of the initial temperature of cell samples. The geometry of M-TUBE used for simulation is 500 μm in diameter and 3 mm in length, and a voltage of 2.50 kV is applied, which leads to an electric field of 8.33 kV/cm. Cell samples are flowed through the microchannel at the same fluid velocity of 148 mm/s.

FIG. 15 is an image of an example high-voltage power supply system used to operate an example embodiment of the M-TUBE. The system is composed of a function generator that allows for waveform programming, a high-voltage amplifier applied to the signal from the function generator, and an oscilloscope that allows for real-time monitoring of the amplified signal.

FIG. 16 is a schematic diagram of the arrangement of electroporation conditions tested in a 96-well deep-well plate. One milliliter of electroporated cells was collected for each combination of electroporation conditions tested. One hundred microliters were dispensed from each 1-mL sample into each of four designated wells containing 900 μL of LB recovery media. For cuvettes experiments, all of the volume aspirated from each cuvette was dispensed into a well.

FIG. 17 is a photograph of an example M-TUBE set up in an anaerobic chamber. The M-TUBE device can be easily and conveniently set up in an anaerobic chamber. The photograph also shows that placing a collection tube (reservoir) directly underneath the fluid as it exits the M-TUBE device would enable the direct and automated transfer of electroporated cells into recovery media.

FIG. 18 is a diagram of a workflow employing a commercial liquid-handling robot for automated liquid transfer and serial dilution. After 1 h of recovery, the 96-well deep plate that contains electroporated samples was mounted on a liquid-handling robot. By leveraging the capabilities of the robot, the M-TUBE device can be used to test a wide range of electroporation conditions, each with at least 3-4 technical replicates, while removing the need for extensive manual pipetting for sample transfer, sample dilution, and sample plating. Strain shown is E. coli NEB10β.

TABLE 2
Residence time (the duration that cells were exposed to electric fields
in M-TUBE devices) as a function of fluid velocities (or flow rates).
M-TUBE device with tubing inner diameter (ID) = 0.5 mm

| | | | | | | | | | | |
|---|---|---|---|---|---|---|---|---|---|---|
| Flow velocity | ~148 | ~296 | ~592 | ~888 | ~1184 | ~1480 | ~1776 | ~2072 | ~2368 | ~2664 |
| (mm/s) | | | | | | | | | | |
| Flow rate | 1.8 | 3.6 | 7.2 | 10.8 | 14.4 | 18.0 | 21.6 | 25.2 | 28.8 | 32.4 |
| (mL/min) | | | | | | | | | | |
| Residence time | ~20.27 | ~10.13 | ~5.07 | ~3.08 | ~2.53 | ~2.03 | ~1.69 | ~1.45 | ~1.27 | ~1.13 |
| (ms) | | | | | | | | | | |

M-TUBE device with tubing ID = 0.8 mm

| | | | | | | | | | | |
|---|---|---|---|---|---|---|---|---|---|---|
| Flow velocity | ~148 | ~296 | ~592 | ~888 | ~1184 | ~1480 | ~1776 | ~2072 | ~2368 | ~2664 |
| (mm/s) | | | | | | | | | | |
| Flow rate | 4.4 | 8.8 | 17.6 | 26.4 | 35.2 | 44.0 | 52.8 | 61.6 | 70.4 | 79.2 |
| (mL/min) | | | | | | | | | | |
| Residence time | ~20.27 | ~10.13 | ~5.07 | ~3.08 | ~2.53 | ~2.03 | ~1.69 | ~1.45 | ~1.27 | ~1.13 |
| (ms) | | | | | | | | | | |

M-TUBE device with tubing ID = 1.6 mm

| | | | | | | | | | | |
|---|---|---|---|---|---|---|---|---|---|---|
| Flow velocity | ~148 | ~296 | ~592 | ~888 | ~1184 | ~1480 | ~1776 | ~2072 | ~2368 | ~2664 |
| (mm/s) | | | | | | | | | | |
| Flow rate | 17.6 | 35.2 | 70.4 | 105.6 | 140.8 | 176.0 | 211.2 | 246.4 | 281.6 | 316.8 |
| (mL/min) | | | | | | | | | | |
| Residence time | ~20.27 | ~10.13 | ~5.07 | ~3.08 | ~2.53 | ~2.03 | ~1.69 | ~1.45 | ~1.27 | ~1.13 |
| (ms) | | | | | | | | | | |

TABLE 3
Comparison of processing times between conventional cuvettes and M-TUBE devices. The processing times
of cuvettes and M-TUBE devices both scale linearly with sample volume. Across all flow velocities
and sample volumes, the M-TUBE device exhibits substantially lower processing time than cuvettes.
0.2-cm cuvette (1-1.5 minutes for 100 μL)

| Processing volume | 1 mL | 5 mL | 10 mL | 50 mL | 100 mL | 500 mL | 1,000 mL |
|---|---|---|---|---|---|---|---|
| Processing time | 10-15 min | 50-75 min | 1.6-2.5 h | 8-12 h | 16-25 h | 83-125 h | 165-250 h |

M-TUBE device (processing time is dependent of flow rates used)

| Processing volume | | 1 mL | 5 mL | 10 mL | 50 mL | 100 mL | 500 mL | 1,000 mL |
|---|---|---|---|---|---|---|---|---|
| M-TUBE | 148 mm/s | 0.56 | 2.78 | 5.56 | 27.78 | 55.56 | 277.78 | 555.56 |
| (Inner diameter | (1.8 mL/min) | | | | | | | |
| (ID) = 0.5 mm) | 296 mm/s | 0.28 | 1.39 | 2.78 | 13.89 | 27.78 | 138.89 | 277.78 |
| Processing | (3.6 mL/min) | | | | | | | |
| time (min) | 592 mm/s | 0.14 | 0.69 | 1.39 | 6.94 | 13.89 | 69.44 | 138.89 |
| | (7.2 mL/min) | | | | | | | |
| | 888 mm/s | 0.09 | 0.46 | 0.93 | 4.63 | 9.26 | 46.30 | 92.59 |
| | (10.8 mL/min) | | | | | | | |
| | 1184 mm/s | 0.07 | 0.35 | 0.69 | 3.47 | 6.94 | 34.72 | 69.44 |
| | (14.4 mL/min) | | | | | | | |
| M-TUBE | 148 mm/s | 0.23 | 1.14 | 2.27 | 11.36 | 22.73 | 113.64 | 227.27 |
| (ID = 0.8 mm) | (4.4 mL/min) | | | | | | | |
| Processing | 296 mm/s | 0.11 | 0.57 | 1.14 | 5.68 | 11.36 | 56.82 | 113.64 |
| time (min) | (8.8 mL/min) | | | | | | | |
| | 592 mm/s | 0.06 | 0.28 | 0.57 | 2.84 | 5.68 | 28.41 | 56.82 |
| | (17.6 mL/min) | | | | | | | |
| | 888 mm/s | 0.04 | 0.19 | 0.38 | 1.89 | 3.79 | 18.94 | 37.88 |
| | (26.4 mL/min) | | | | | | | |
| | 1184 mm/s | 0.03 | 0.14 | 0.28 | 1.42 | 2.84 | 14.20 | 28.41 |
| | (35.2 mL/min) | | | | | | | |
| M-TUBE | 148 mm/s | 0.06 | 0.28 | 0.57 | 2.84 | 5.68 | 28.41 | 56.82 |
| (ID = 1.6 mm) | (17.6 mL/min) | | | | | | | |
| Processing | 296 mm/s | 0.03 | 0.14 | 0.28 | 1.42 | 2.84 | 14.20 | 28.41 |
| time (min) | (35.2 mL/min) | | | | | | | |
| | 592 mm/s | 0.01 | 0.07 | 0.14 | 0.71 | 1.42 | 7.10 | 14.20 |
| | (70.4 mL/min) | | | | | | | |
| | 888 mm/s | 0.01 | 0.05 | 0.09 | 0.47 | 0.95 | 4.73 | 9.47 |
| | (105.6 mL/min) | | | | | | | |
| | 1184 mm/s | 0.01 | 0.04 | 0.07 | 0.36 | 0.71 | 3.55 | 7.10 |
| | (140.8 mL/min) | | | | | | | |

TABLE 4
Comparison of costs for assembly of one M-TUBE device
versus cuvettes per unit processing volume.
Parts cost for M-TUBE devices and conventional cuvettes

| Parts for M- | | Bulk Price | |
|---|---|---|---|
| TUBE | Quantity | (USD) | Note |
| Syringe needle | 1000 pieces | <$101.9 | (<$0.10 per piece) |
| Plastic tubing | 3048 cm | <$58.9 | (<$0.02 per cm) |
| | | | (One M-TUBE |
| | | | device costs <$0.22) |
| Parts for | | Bulk Price | |
| cuvettes | Quantity | (USD) | Note |
| 0.2-cm cuvette | 50 pieces | $111.38 | ($2.23 per cuvette) |
| from VWR | | | |
| 0.2-cm cuvette | 50 pieces | $117.75 | ($2.36 per cuvette) |
| from BIO-RAD | | | |

Cost to electroporate a unit volume sample (parts only)

| | | | |
|---|---|---|---|
| Processing | 0.2-cm cuvette | 0.2-cm cuvette | M-TUBE |
| volume | from VWR | from BIO-RAD | device |

| | | | | |
|---|---|---|---|---|
| 1 | mL | $22.30 | $23.60 | <$0.22 |
| 5 | mL | $111.38 | $117.75 | <$0.22 |
| 10 | mL | $222.76 | $235.50 | <$0.22 |
| 50 | mL | $1,113.80 | $1,177.50 | <$0.22 |
| 100 | mL | $2,227.60 | $2,355.00 | <$0.22 |
| 500 | mL | $11,138.00 | $11,775.00 | <$0.22 |
| 1000 | mL | $22,276.00 | $23,550.00 | <$0.22 |

TABLE 5
Strains, plasmids, and oligos used in this study.

| Strain | | Source | |
|---|---|---|---|
| E. coli NEB10β | | New England Biolabs | |
| E. coli K-12 MG1655 | | Yale Coli Genetics Stock Center | |
| E. coli Nissle 1917 | | Mutaflor® | |
| Bifidobacterium longum | | Gift from Douwe van Sinderen, University | |
| subsp. longum NCIMB8809 | | College Cork, Ireland | |
| Plasmid | Reference | Source | |
| pCon1.00 | http://parts.igem.org/Part:BBa_K176011 | iGEM | |
| (J23100)- | | | |
| >RBS + | | | |
| GFP + T | | | |
| pAM5 | [44] | Gift from Douwe van Sinderen, University | |
| | | College Cork, Ireland | |
| Oligo | Sequence | Description | Source |
| Erm-For | /5Phos/CTGTCTCTTATACACATCTATTT | Forward primer for | This study |
| | ATGTTACAGTAATATTGACTTCGACACC | generating randomly | |
| | [SEQ ID NO: 1] | barcoded erm^{R} transposon | |
| Erm-Rev | /5Phos/CTGTCTCTTATACACATCT | Reverse primer for generating | This study |
| | GTCGACCTGCAGCGTACG | randomly barcoded erm^{R} | |
| | NNNNNNNNNNNNNNNNNNNN | transposon | |
| | AGAGACCTCGTGGACATC | | |
| | TTACACATTATTCCGGTGATAGGGC | | |
| | [SEQ ID NO: 2] | | |
| Adaptor-A | /5Phos/GATCGGAAGAGCACACGTCTGAACTCCA | First half of adaptor for Tn- | Reference [33] |
| | GTCA | seq library preparation | |
| | [SEQ ID NO: 3] | | |
| Adaptor-B | ACGCTCTTCCGATC*T | Second half of adaptor for Tn- | Reference [33] |
| | [SEQ ID NO: 4] | seq library preparation. ‘*’ | |
| | | mark is shorthand for a | |
| | | phosphorothioate bond. | |
| Tnseq-For | ATGATACGGCGACCACCGAGATCTACACTCTTTCC | Forward primer for Tn-seq | Reference [33] |
| | CTACACGACGCTCTTCCGATCT NNNNNN | library amplification, Tn5 | |
| | GATGTCCACGAGGTCT | binding, introduces P5 | |
| | [SEQ ID NO: 5] | | |
| Tnseq-Rev | CAAGCAGAAGACGGCATACGAGAT ATTGGC | Reverse primer for Tn-seq | Reference [33] |
| | GTGACTGGAGTTCAGACGTGTGCTCTTCCGATCT | library amplification, adaptor | |
| | [SEQ ID NO: 6] | binding, introduces P7 | |

References

1. Mazodier P. Davies J. Gene-Transfer between Distantly Related Bacteria. Annu Rev Genet. 1991;25:147-71. doi: DOI 10.1146/annurev.ge.25.120191.001051. PubMed PMID: WOS: A1991GX17400007.

2. Yoshida N, Sato M Plasmid uptake by bacteria: a comparison of methods and efficiencies. Applied Microbiology and Biotechnology. 2009;83(5):791-8. doi: 10.1007/s00253-009-2042-4. PubMed PMID: WOS 000267214600001.

3. Rivera AL, Magana-Ortiz D, Gomez-Lim M, Fernandez F, Loske AM Physical methods for genetic transformation of fungi and yeast. Phys Life Rev. 2014; 11(2): 184-203. doi: 10.1016/j.plrev 2014 01.007 PubMed PMID: WOS:000337982800007.

4. Sheng Y, Mancino V, Birren B Transformation of Escherichia-Coli with Large DNA-Molecules by Electroporation. Nucleic Acids Research. 1995;23(11): 1990-6. PubMed PMID: WOS:A1995RE99600023

5. Chassy BM, Mercenier A, Flickinger J. Transformation of Bacteria by Electroporation. Trends Biotechnol. 1988;6(12):303-9. doi: Doi 10.1016/0167-7799(88)90025-X. PubMed PMID: WOS:A1988R036000007.

6. Kotnik T, Frey W, Sack M, Meglic SH, Peterka M, Miklavcic D Electroporation-based applications in biotechnology. Trends Biotechnol 2015;33(8):480-8. doi: 10.1016/j.tibtech 2015 06.002 PubMed PMID: WOS 000358805500009.

7. Neumann E. Membrane Electroporation and Direct Gene-Transfer. Bioelectroch Biocner. 1992;28(1-2):247-67. doi: Doi 10.1016/0302-4598(92)80017-B PubMed PMID: WOS: A1992JK13500018.

8. Shiver AL, Culver R, Deutschbauer AM, Huang KC Rapid ordering of barcoded transposon insertion libraries of anaerobic bacteria. Nature Protocols. 2021;16(6):3049- +. PubMed PMID WOS 000652922800002

9. Hess GT, Fresard L, Han K, Lee CH, Li A, Cimprich KA, et al. Directed evolution using dCas9-targeted somatic hypermutation in mammalian cells. Nature Methods. 2016;13(12): 1036-+. doi: 10.1038/Nmeth.4038. PubMed PMID: WOS:000389133000024.

10. Ruiz L, Bottacini F, Boinett CJ, Cain AK, O'Conncll-Mothenvay M, Lawley TD, et al. The essential genomic landscape of the commensal Bifidobacterium breve UCC2003. Scientific Reports. 2017;7. doi: ARTN 5648

10.1038/s41598-017-05795-y. PubMed PMID: WOS:000405677200033.

11. Movahed S, Li DQ Microfluidicscell electroporation Microfluid Nanofluid 2011;10(4):703-34. doi: 10.1007/s 10404-010-0716-y PubMed PMID: WOS:000288254600001

12. Geng T, Lu C. Microfluidic electroporation for cellular analysis and delivery. Royal Society of Chemistry; 2013 p. 3803-21.

13. Gac SL, Uitert Iv. Electroporation in Microfluidic Devices. Handbook of Electroporation. 2016:1-20. doi: 10.1007/978-3-319-26779-1_136-1.

14. Fox MB, Esveld DC, Valero A, Luttge R, Mastwijk HC, Bartels PV, et al. Electroporation of cells in microfluidic devices: a review. Anal Bioanal Chem 2006;385(3):474-85. Epub 2006/03/15. doi: 10.1007/s00216-006-0327-3. PubMed PMID: 16534574.

15. Wang HY, Lu C. Electroporation of mammalian cells in a microfluidic channel with geometric variation. Analytical Chemistry. 2006;78(14):5158-64. doi: 10.1021/ac060733n

16. Wang H-Y, Lu C. Microfluidic electroporation for delivery of small molecules and genes into cells using a common DC power supply. Biotechnology and Bioengineering. 2008; 100(3):579-86. doi: 10 1002/bit 21784

17. Ziv R. Steinhardt Y, Pelled G. Gazit D, Rubinsky B. Micro-electroporation of mesenchymal stem cells with alternating electrical current pulses. Biomedical Microdcvices. 2009;11:95-101 doi 10.1007/s 10544-008-9213-4 PubMed PMID 18815886

18. Geng T, Zhan Y, Wang J, Lu C. Transfection of cells using flow-through electroporation based on constant voltage. Nature Protocols. 2011;6(8): 1192-208. doi: 10.1038/nprot.2011.360.

19. Garcia PA, Ge Z, Kelley LE, Holcomb SJ, Buie CR High efficiency hydrodynamic bacterial electrotransformation Lab on a Chip. 2017;17:490-500. doi 10.1039/c61c01309k PubMed PMID: 28067371

20. Guignet EG, Meyer T. Suspendec-drop electroporation for high-throughput delivery of biomolecules into cells. Nature Methods. 2008;5(5):393-5. doi: 10.1038/nmeth.1201.

21. Madison AC, Royal MW, Vigneault F, Chen L, Griffin PB, Horowitz M, et al Scalable Device for Automated Microbial Electroporation in a Digital Microfluidic Platform. ACS Synthetic Biology. 2017;6(9): 1701-9 doi: 10.1021/ACSSYNBIO 7B00007

22. Hamieh M. Dobrin A, Cabriolu A, van der Stegen SJC, Giavridis T, Mansilla-Soto J, et al. CAR T cell trogocytosis and cooperative killing regulate tumour antigen escape Nature. 2019;568(7750): 112-+doi 10.1038/s41586-019-1054-1 PubMed PMID WOS:000463384900050.

23. Feucht J, Sun J, Eyquem J, Ho YJ, Zhao ZG, Leibold J, et al. Calibration of CAR activation potential directs alternative T cell fates and therapeutic potency (vol 25, pg 82, 2019). Nat Med. 2019;25(3):530-. doi: 10.1038/s41591-019-0360-3. PubMed PMID: WOS:000460643100036

24. Eyquem J, Mansilla-Soto J, Giavridis T, van der Stegen SJC, Hamieh M, Cunanan KM, et al. Targeting a CAR to the TRAC locus with CR1SPR/Cas9 enhances tumour rejection. Nature. 2017,543(7643):113-+. doi: 10.1038/nature21405. PubMed PMID: WOS:000395671500043.

25. Dever DP, Bak RO, Reinisch A, Camarena J, Washington G, Nicolas CE, et al. CRISPR/Cas9 beta-globin gene targeting in human haematopoietic stem cells. Nature. 2016;539(7629):384-9. doi: 10. 1038/nature20134 PubMed PMID: WOS:000388I61700045.

26. Bozza M, De Roia A, Correia MP, Berger A, Tuch A, Schmidt A, et al A nonviral, nonintegrating DNA nanovector platform for the safe, rapid, and persistent manufacture of recombinant T cells. Science Advances. 2021;7(16). doi: ARTN eabf1333

10.1126/sciadv.abf1333. PubMed PMID: WOS:000640217000013.

27. Sonnenborn U. Escherichia coli strain Nissle 1917-from bench to bedside and back: history of a special Escherichia coli strain with probiotic properties. Ferns Microbiol Lett. 2016;363(19). doi: ARTN fnw212

10.1093/femsle/fnw212. PubMed PMID: WOS 000388381800006

28. Praveschotinunt P, Duraj-Thatte AM, Gelfat I, Bahl F, Chou DB, Joshi NS. Engineered IC. co/i Nissle 1917 for the delivery of matrix-tethered therapeutic domains to the gut. Nat Commun. 2019;10. PubMed PMID: WOS:090502072000001.

37. Gallo-Villanueva RC, Sano MB, Lapizco-Encinas BH, Davalos RV. Joule heating effects on particle immobilization in insulator-based dielectrophoretic devices. Electrophoresis. 2014;35(2-3):352-61. Epub 2013/09/05. doi: 10.1002/elps 201300171 PubMed PMID: 24002905; PubMed Central PMCID: PMCPMC4114348

38. Silve A, Leray I, Poignard C, MirLM. Impact of external medium conductivity on cell membrane electropermeabilization by microsecond and nanosecond electric pulses. Sci Rep. 2016;6; 19957. Epub 2016/02/02. doi; 10.1038/srep 19957. PubMed PMID; 26829153; PubMed Central PMCID; PMCPMC4734290.

39. Garcia PA, Rossmeisl JH, Jr., Neal RE, 2nd, Ellis TL, Olson JD, Henao-Guerrero N, et al. Intracranial nonthermal irreversible electroporation: in vivo analysis. J Membr Biol. 2010;236(1)127-36. Epub 2010/07/30 doi 10.1007/s00232-010-9284-z. PubMed PMID 20668843

40. Sel D, Cukjati D. Batiuskaite D, Slivnik T, Mir LM, Miklavcic D. Sequential finite element model of tissue electropermeabilization. IEEE Trans Biomcd Eng. 2005;52(5):816-27. Epub 2005/05/13. doi: 10.1109/TBME.2005.845212. PubMed PMID: 15887531.

41. O'Callaghan A, Bottacini F, O'Connell Motherway M, van Sinderen D Pangenome analysis of Bifidobacterium longum and site-directed mutagenesis through by-pass of restriction-modification systems. BMC Genomics. 2015; 16:832. Epub 2015/10/23. doi: 10.1186/s 12864-015-1968-4. PubMed PMID: 26489930; PubMed Central PMCID: PMCPMC4618763.

42. Park MJ, Park MS, Ji GE. Improvement of electroporation-mediated transformation efficiency for a Bifidobacterium strain to a reproducibly high level. J Microbiol Methods. 2019; 159:112-9 Epub 2018/12/12. doi: 10.1016/j mimet 2018.11.019 PubMed PMID: 30529116.

43. Ruiz L, Motherway MO. Lanigan N, van Sinderen D Transposon mutagenesis in Bifidobacterium breve: construction and characterization of a Tn5 transposon mutant library for Bifidobacterium breve UCC2003. PLoS One. 2013;8(5):e64699. Epub 2013/06/06. doi: 10.1371/journal.pone.0064699. PubMed PMID: 23737995; PubMed Central PMCID: PMCPMC3667832.

44. Alvarez-Martin P, O'Connell-Moiherway M, van Sindercn D, Mayo B. Functional analysis of the pBCl replicon from Bifidobacterium catenulatum L48. Appl Microbiol Biotechnol. 2007;76(6): 1395-402. Epub 2007/08/21. doi: 10.1007/s00253-007-1 115-5. PubMed PMID: 17704917.

What is claimed is:

1. An electroporation device, comprising:
at least two conductive elements, each of the at least two conductive elements being of a hollow, tubular structure; and
an insulating structure defining a channel of constant diameter, the insulating structure configured to fluidically couple the at least two conductive elements, the at least two conductive elements and the insulating structure in coupled arrangement defining an electroporation flow path in the channel for flow-through electroporation.

2. The electroporation device of claim 1, wherein each of the at least two conductive elements is a cannula comprising a conductive material.

3. The electroporation device of claim 1, wherein each of the at least two conductive elements is a syringe needle.

4. The electroporation device of claim 1, wherein the insulating structure is a polymer tube.

5. The electroporation device of claim 1, wherein the insulating structure is configured to receive the at least two conductive elements as inserts at opposing ends of the channel.

6. The electroporation device of claim 5, wherein the insulating structure, or the at least two conductive elements, includes markings indicating an insertion distance of each the at least two conductive elements.

7. The electroporation device of claim 5, wherein the insulating structure, or the at least two conductive elements, includes stops defining an insertion distance of each of the at least two conductive elements.

8. The electroporation device of claim 5, wherein the at least two conductive elements are inserted into the channel of the insulating structure with a gap therebetween of about 1 mm to about 50 mm, or of about 1 mm to about 10 mm.

9. The electroporation device of claim 1, further comprising a fluid pump coupled to an upstream one of the at least two conductive elements.

10. The electroporation device of claim 9, wherein the fluid pump is configured to supply a cell media to the channel at a flow rate of about 1 mL/min to about 1500 mL/min, or of about 1 mL/min to about 100 mL/min.

11. The electroporation device of claim 9, further comprising a controller configured to control the flow rate based upon a selected residence time of cells exposed to an electric field in the channel.

12. The electroporation device of claim 1, further comprising a power supply in operative arrangement with the at least two conductive elements.

13. The electroporation device of claim 12, wherein a voltage supplied by the power supply is configured to generate an electric field within the channel of about 0.1 kV/cm to about 100 kV/cm.

14. The electroporation device of claim 12 further comprising a controller configured to control an applied voltage based upon a selected electric field strength.

15. The electroporation device of claim 14, wherein control of the applied voltage is further based on at least one of a channel diameter and a channel distance.

16. The electroporation device of claim 12, further comprising an indicator configured to indicate an applied current in the flow path.

17. The electroporation device of claim 1, wherein the channel defined by the insulating structure has a diameter of about 0.1 mm to about 5 mm.

18. The electroporation device of claim 1, wherein the insulating structure is disposable.

19. The electroporation device of claim 1, wherein the at least two conductive elements are disposable.

20. The electroporation device of claim 1, wherein the channel is configured to enable fluid to travel through the electroporation flow path at an average velocity of about 0.1 m/s to about 5 m/s.

21. The electroporation device of claim 1, wherein the channel is configured to enable fluid to travel through the electroporation flow path at a constant average velocity.

22. A method of fabricating an electroporation device, comprising:
inserting a conductive element at each opposing end of an insulating structure defining a channel of constant diameter, each conductive element being of a hollow, tubular structure, the conductive elements and the insulating structure in coupled arrangement defining an electroporation flow path in the channel for flow-through electroporation.

23. A kit comprising:
a plurality of conductive elements, each of the conductive elements being of a hollow, tubular structure; and
a plurality of insulating structures, each insulating structure defining a channel of constant diameter and configured to fluidically couple at least two of the plurality of conductive elements, the at least two conductive elements and the insulating structure in coupled arrangement defining an electroporation flow path in the channel for flow-through electroporation.


SEQUENCE LISTING (6 sequences)

SEQ ID NO: 1 - DNA, 51 bp, synthetic construct
  misc_feature 1..51: note = Erm-For
  source 1..51: mol_type = other DNA
  misc_feature 1: note = 5Phos
ctgtctcttatacacatctatttatgttacagtaatattgacttcgacacc

SEQ ID NO: 2 - DNA, 100 bp, synthetic construct
  misc_feature 1..100: note = Erm-Rev
  variation 38..57: note = n is a, c, g, or t
  source 1..100: mol_type = other DNA
  misc_feature 1: note = 5Phos
ctgtctcttatacacatctgtcgacctgcagcgtacgnnnnnnnnnnnnnnnnnnnnagagacctcgtggacatcttacacattattccggtgatagggc

SEQ ID NO: 3 - DNA, 32 bp, synthetic construct
  misc_feature 1..32: note = Adaptor-A
  source 1..32: mol_type = other DNA
  misc_feature 1: note = 5Phos
gatcggaagagcacacgtctgaactccagtca

SEQ ID NO: 4 - DNA, 15 bp, synthetic construct
  misc_feature 1..15: note = Adaptor-B
  source 1..15: mol_type = other DNA
  modified_base 14^15: mod_base = OTHER; note = phosphorothioate bond
acgctcttccgatct

SEQ ID NO: 5 - DNA, 79 bp, synthetic construct
  misc_feature 1..79: note = Tnseq-For
  variation 58..63: note = n is a, c, g, or t
  source 1..79: mol_type = other DNA
atgatacggcgaccaccgagatctacactctttccctacacgacgctcttccgatctnnnnnngatgtccacgaggtct

SEQ ID NO: 6 - DNA, 64 bp, synthetic construct
  misc_feature 1..64: note = Tnseq-Rev
  source 1..64: mol_type = other DNA
caagcagaagacggcatacgagatattggcgtgactggagttcagacgtgtgctcttccgatct